(19) United States
(12) Patent Application Publication
(10) Pub. No.: US 2004/0235053 A1
(43) Pub. Date: Nov. 25, 2004

(54) Preparation and application of encoded bead aggregates in combinatorial chemistry

(75) Inventors: Kit S. Lam, Davis, CA (US); Jan Marik, Sacramento, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/811,243

(22) Filed: Mar. 25, 2004

Related U.S. Application Data
(60) Provisional application No. 60/458,252, filed on Mar. 28, 2003.

(51) Int. Cl. C12Q 1/68; G01N 33/53; G01N 33/543

(52) U.S. Cl. 435/7.1; 436/518

(57) ABSTRACT

The present invention relates to methods of preparing a library of compounds using encoded bead aggregates. The structural features of the compounds are encoded, and the quantities of compound prepared are sufficient for solution phase studies.

### CROSS-REFERENCE TO RELATED APPLICATION

This application claims the benefit of U.S. Provisional Application Serial No. 60/458,252, filed Mar. 28, 2003, the content of which is incorporated herein by reference.

### STATEMENT AS TO RIGHTS TO INVENTIONS MADE UNDER FEDERALLY SPONSORED RESEARCH AND DEVELOPMENT

A portion of the present invention was made under federally sponsored research and development under National Institutes of Health/National Cancer Institute Grant No. R33 CA 89706. The Government may have rights in certain aspects of this invention.

### BACKGROUND OF THE INVENTION

The split-mix synthesis method (Lam, K. S. et al. Nature 1991, 354, 82-84; Houghten, R. A. et al. Nature 1991, 354, 84-86; Furka, A. et al. Int. J Peptide Protein Res. 1991, 37, 487-493) enables one to efficiently generate thousands to millions of chemical compounds, such that each bead displays only one compound entity, and there are 10¹³ copies of the same compound on one single bead. This “one-bead one-compound” (OBOC) concept first recognized by Lam (Lam, K. S. et al. Nature 1991, 354, 82-84) has enabled the screening of libraries in an ultra-high throughput fashion using an on-bead screening assay. Literally millions of compounds can be screened in a matter of a few days. Many ligands or substrates for a number of biological targets have been discovered with this approach (Lam, K. S., et al. Chem. Rev. 1997, 97, 411-448). However, the successful use of the OBOC combinatorial libraries in a solution phase screening assay has been limited, because of the small amount of compound bound to each bead. Even with macrobeads, no more than 0.1 μmol of material can be recovered from one single bead (Blackwell, H. E., et al. Chem Biol 2001, 8, 1167-1182; Clemons, P. A., et al. Chem Biol 2001, 8, 1183-1195). Therefore to improve the capabilities of the OBOC concept, an inexpensive solid support is needed that (i) has significantly higher capacity than a macrobead, and (ii) can be easily encoded and decoded. Surprisingly, the present invention meets this and other needs.

### SUMMARY OF THE INVENTION

The present invention provides methods for preparing a library of encoded compounds, such that a sufficient quantity of compound is prepared so that solution phase studies can be performed. The novel feature of this method is the use of an aggregate of crosslinked beads for the preparation of the compounds. This bead aggregate comprises two types of beads, a compound bead and a coding bead, with a high percentage of compound beads. Following preparation of the compound library, the compounds are cleaved from the compound beads for subsequent screening, and the coding sequence is analyzed on the coding bead to decode the compound.

In one aspect, the present invention provides a method for preparing a library of compounds, comprising: a) providing a plurality of individual bead aggregates, wherein each of the bead aggregates comprises a population of compound beads and a population of coding beads, wherein the compound beads and the coding beads are crosslinked to each other, wherein each of the compound beads comprises a scaffold linked to the compound bead via a scaffold linker, and with at least two scaffold functional groups attached to the scaffold, and wherein each of the coding beads comprises at least one coding functional group; b) contacting a first bead aggregate with a first reactive component such that a first scaffold functional group reacts with the first reactive component to afford a first scaffold building block; c) contacting the first bead aggregate with a successive reactive component such that a subsequent scaffold functional group reacts with the successive reactive component to afford a subsequent scaffold building block; d) repeating step c) until the first compound has been prepared; and e) subjecting additional bead aggregates to steps b)-d) with additional reactive components to prepare the library of compounds.

In another aspect, the present invention provides a method for preparing a library of compounds via the split-mix methodology, comprising: a) providing a plurality of individual bead aggregates, wherein each of the bead aggregates comprises a population of compound beads and a population of coding beads, wherein the compound beads and the coding beads are crosslinked to each other, wherein each of the compound beads comprises a scaffold linked to the compound bead via a scaffold linker, and with at least two scaffold functional groups attached to the scaffold, and wherein each of the coding beads comprises at least one coding functional group; b) splitting the bead aggregates into two or more separate pools; c) contacting the bead aggregates with one or more first reactive components in the two or more separate pools such that a first scaffold functional group reacts with one of the first reactive components to afford a first scaffold building block, wherein the contacting step affords subsequent bead aggregates; d) encoding each of the scaffold building blocks with a coding building block, comprising the step of contacting the coding functional group with a reactive component such that the coding functional group reacts with the reactive component to afford a coding building block linked to the coding bead, wherein the coding building block encodes one of the scaffold building blocks, and wherein the encoding step yields subsequent encoded bead aggregates; e) mixing the subsequent encoded bead aggregates from the two or more separate pools into a single pool; f) splitting the subsequent encoded bead aggregates into two or more separate pools; g) contacting the subsequent encoded bead aggregates in the two or more separate pools with a successive reactive component such that a subsequent scaffold functional group reacts with the successive reactive component to afford a subsequent scaffold building block, wherein the contacting step yields further bead aggregates; h) repeating step d), wherein the encoding step yields further encoded bead aggregates; and i) repeating steps e)-h), wherein the further encoded bead aggregates of step h) become the subsequent encoded bead aggregates of step e), until the library of compounds has been prepared.

### BRIEF DESCRIPTION OF THE DRAWINGS

FIG. 1. Schematic showing the stepwise preparation of a compound of a library of the present invention, and concomitant encoding of the building block of each reaction. Following preparation of the compound, the compound is cleaved, the coding sequence is analyzed and the compound decoded. The sequential encoding methodology is exemplified.

FIG. 2. Schematic showing the stepwise preparation of a compound of a library of the present invention, and concomitant encoding of the building block of each reaction. The separately attached encoding methodology is exemplified.

### DETAILED DESCRIPTION OF THE INVENTION

I. Definitions

As used herein, the term “library of compounds” refers to a collection of compounds on separate phase support particles in which each separate phase support particle contains a single structural species of the synthetic test compound. Each support contains many copies of the single structural species.

As used herein, the term “compound” refers to a small molecule, peptide, peptoid, polyketide, etc., consisting of 2 to 100, and more preferably, 2-20, functional groups, with or without a scaffold. In one embodiment, the compound is an aromatic heterocycle with three functional groups.

As used herein, the term “bead aggregate” refers to an agglomeration of beads that are interconnected to one another to form a single structure. In the present invention, a bead aggregate is comprised of several hundreds or thousands of compound beads and coding beads that are crosslinked to one another.

As used herein, the term “compound bead” refers to a solid phase support that will be used to prepare a compound.

As used herein, the term “coding bead” refers to a solid phase support of the present invention where the coding of the scaffold building blocks occurs.

As used herein, the term “crosslinked” refers to the state of having numerous solid phase supports interconnected to each other such that they become a single structure. The chemical functionality that links the individual solid phase supports that are crosslinked, is termed a “crosslinker”. A crosslinker is typically a bifunctional compound that reacts with one reactive functional group on one solid phase support and one reactive functional group on another sold phase support, thereby linking the two solid phase support members to each other. In a preferred embodiment, the individual solid phase support members of the present invention are attached to at least one other solid phase support member. The preferred crosslinkers of the present invention are stable to the reaction conditions for the preparation and encoding of the compound.

As used herein, the term “scaffold” refers to a structure which can be a cyclic or bicyclic hydrocarbon, a steroid, a sugar, a heterocyclic structure, a polycyclic aromatic molecule, an amine, an amino acid, a multi-functional small molecule, a peptide or a polymer, having various substituents at defined positions. Preferred scaffolds of the present invention include, but are not limited to, quinazoline, quinoxaline, purine, pyrimidine, phenyl, naphthyl, indole, benzimidazole, phthalazine, tertiary amine, triazine, quinoline, coumarin, amino acid and peptide. Scaffolds of the present invention also include a single atom, such as carbon or nitrogen.

As used herein, the term “scaffold linker” refers to a chemical moiety that links the scaffold to the solid phase support. Scaffold linkers of the present invention, include, but are not limited to, aminobutyric acid, aminocaproic acid, 7-aminoheptanoic acid, 8-aminocaprylic acid, lysine, iminodiacetic acid, polyoxyethylene, glutamic acid, etc. In a further embodiment, linkers of the present invention can additionally comprise one or more β-alanines or other amino acids as spacers.

As used herein, the term “scaffold functional group” refers to a chemical moiety that is a precursor to the corresponding scaffold building block. Preferred scaffold functional group include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. One of skill in the art will be aware of other common functional groups that are encompassed by the present invention.

As used herein, the term “contacting” refers the process of bringing into contact at least two distinct species such that they can react. In one embodiment, contacting an amine and an ester under conditions known to one of skill in the art would result in the formation of an amide.

As used herein, the term “reactive component” refers to a chemical or reagent being used to modify a functional group into a building block.

As used herein, the term “scaffold building block” refers to a chemical moiety that has been transformed by reacting a scaffold functional group with a reactive component.

As used herein, the term “cleaving” refers to the breaking of a bond or a connecting element of the present invention.

As used herein, the terms “encode”, “encoded” and “encoding” refer to a library of compounds in which each distinct species of compound is paired on each separate solid phase support with at least one coding building block containing a functional group that is the same or mimics a particular functional group of the compound. In one embodiment, there is one coding building block for each functional group on the compound.

As used herein, the term “coding” is used as a prefix denoting that a particular feature or item is a part of the mechanism that encodes each functional group of the compounds in the library.

As used herein, the term “coding functional group” refers to a chemical moiety that is a precursor to the corresponding coding building block. Preferred coding functional group include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. One of skill in the art will be aware of other common functional groups that are encompassed by the present invention. A coding functional group of the present invention can already be a part of the coding bead, or can be subsequently added on to the coding bead.

As used herein, the term “coding building block” refers to a chemical moiety that has been transformed by reacting a coding functional group with a reactive component. The coding building block encodes the chemical functionality of the corresponding scaffold building block.

As used herein, the term “coding linker” refers to a chemical moiety that optionally connects the coding functional group to the solid phase support. The coding linker also optionally connects the coding building block to the solid phase support. Coding linkers of the present invention, include, but are not limited to, aminobutyric acid, aminocaproic acid, 7-aminoheptanoic acid, 8-aminocaprylic acid, lysine, iminodiacetic acid, polyoxyethylene, glutamic acid, etc. In a further embodiment, linkers of the present invention can additionally comprise one or more β-alanines or other amino acids as spacers.

As used herein, the term “interior portion” refers to that portion of the solid phase support that substantially excludes the surface of the solid phase support.

As used herein, the term “exterior portion” refers to that portion of the solid phase support that substantially includes the surface of the solid phase support.

As used herein, the term “coding sequence” refers to a set of coding building blocks that are separately attached to the solid support and encode the corresponding scaffold building blocks attached to the same solid support, or to a set of coding building blocks that are sequentially linked to the coding bead. In a preferred embodiment, coding sequence refers to a set of coding building blocks that are sequentially linked to the solid support and encode the corresponding scaffold building blocks attached to the same solid support.

As used herein, the term “mixing” refers to the act of combining individual elements such that they cannot be easily distinguished or separated.

II. General

As combinatorial chemistry has become an indispensable part of compound synthesis and drug discovery, the split-mix methodology has become an essential tool. While the split-mix methodology is advantageous due to its rapid and facile encoding and screening of the compounds generated, the method is not readily amenable to solution phase screening due to the minute amount of compound generated. The present invention provides a method for preparing a library of compounds that generates quantities of compound that are suitable for conventional solution phase screening and repeating assays. The bead aggregates of the present invention are comprised of two types of beads, compound beads and coding beads, that are crosslinked together. By keeping the percentage of coding beads small, the number of beads containing the compounds of the library is greatly increased. Following preparation of the compound, the compound is cleaved from the beads, and the coding beads are analyzed in order to decode the compound.

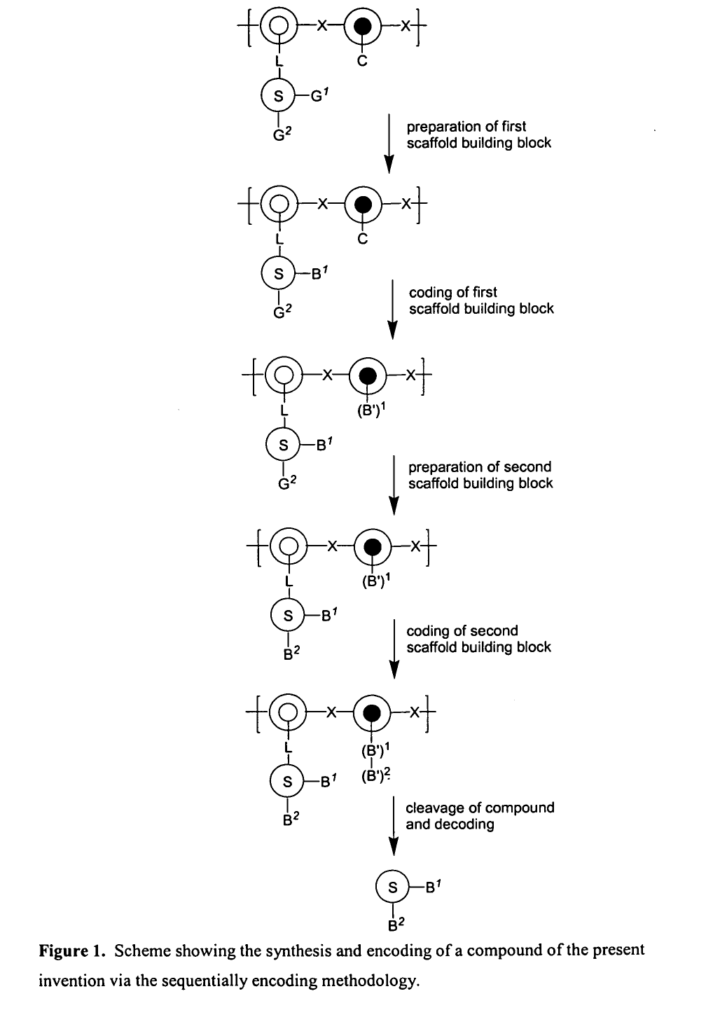

Using the bead aggregates, the compounds of the present invention are prepared on the compound beads and are subsequently encoded on the coding beads. FIG. 1 shows a bead aggregate comprising a compound bead (light circle) and a coding bead (darkened circle) crosslinked via crosslinker X. Attached to the compound bead is a scaffold (S) with two scaffold functional groups (G¹ and G²). The scaffold is attached to the compound bead via a scaffold linker (L). Attached to the coding bead is a coding functional group (C). As FIG. 1 demonstrates, the bead aggregate is subjected to a first set of reaction conditions, converting the first scaffold functional group (G¹) to the first scaffold building block (B¹). The first scaffold building block is then encoded with a first coding building block ((B′)¹) on the coding bead. The second scaffold functional group (G²) is subsequently converted to the second scaffold building block (B²), which is then encoded with the second coding building block ((B′)²) on the coding bead. The second coding building block is attached to the coding bead through the first coding building block, and subsequent coding building blocks are attached to the previous coding building block. In this manner, the coding building blocks create the coding sequence. When the compound has been prepared, it is cleaved from the compound beads, and the coding sequence is then analyzed in order to decode the compound.

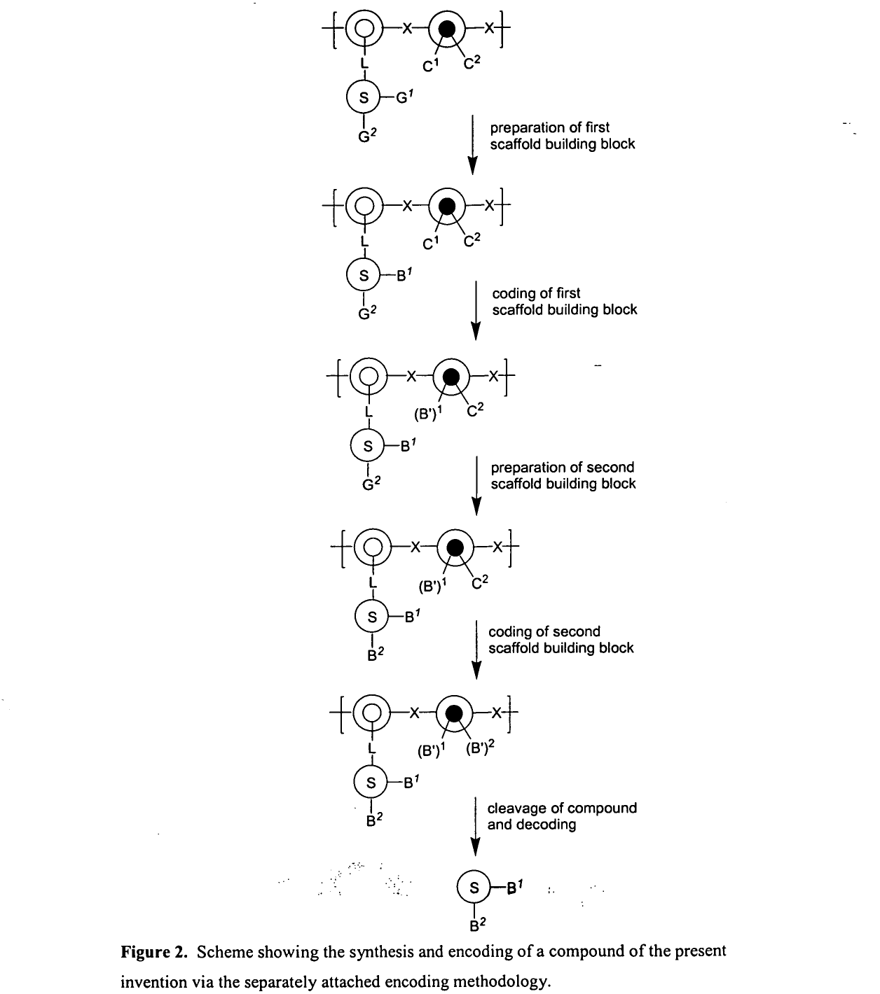

Alternatively, there are at least two coding functional groups, each separately attached to the coding bead (C¹ and C²). As described above, each scaffold building block is prepared separately, and subsequently encoded in a separate step with a coding building block ((B′)¹ and (B′)²). In the separately attached encoding methodology, the coding building blocks are separately attached to the coding bead, as shown in FIG. 2.

III. Method for the Preparation of Encoded Bead Aggregate Libraries

In one aspect, the present invention provides a method for preparing a library of compounds, comprising: a) providing a plurality of individual bead aggregates, wherein each of the bead aggregates comprises a population of compound beads and a population of coding beads, wherein the compound beads and the coding beads are crosslinked to each other, wherein each of the compound beads comprises a scaffold linked to the compound bead via a scaffold linker, and with at least two scaffold functional groups attached to the scaffold, and wherein each of the coding beads comprises at least one coding functional group; b) contacting a first bead aggregate with a first reactive component such that a first scaffold functional group reacts with the first reactive component to afford a first scaffold building block; c) contacting the first bead aggregate with a successive reactive component such that a subsequent scaffold functional group reacts with the successive reactive component to afford a subsequent scaffold building block; d) repeating step c) until the first compound has been prepared; and e) subjecting additional bead aggregates to steps b)-d) with additional reactive components to prepare the library of compounds.

The libraries of compounds of the present invention are prepared using bead aggregates which are comprised of compound beads and coding beads that are crosslinked to one another and each other. The compound beads of the present invention comprise a scaffold linked to the interior of the compound bead via a scaffold linker, wherein the scaffold comprises at least two scaffold functional groups. The exterior reactive functional groups of the compound beads are used for linking to the crosslinker. The coding beads of the present invention comprise two types of reactive functional groups: exterior and interior reactive functional groups. The exterior reactive functional groups are useful for linking to the crosslinker, while the interior reactive functional groups link to the coding sequence. One of skill in the art will recognize that other components may be incorporated.

Libraries of the present invention include libraries of compounds bound to a solid support, as well as libraries of compounds that are not bound to a solid support. In a preferred embodiment, the present invention provides a library of compounds bound to a solid support and prepared by the method described above. In another preferred embodiment, the method of the present invention further comprises the following step: f) cleaving each of the compounds from each of the bead aggregates. In yet another preferred embodiment, the present invention provides a library of compounds wherein the compounds are not bound to a solid support.

A. Encoding the Building Blocks of the Compound

In a further embodiment, the method of the present invention comprises the step of encoding each of the scaffold building blocks with a coding building block. In yet another embodiment, each the scaffold building blocks is encoded with one of the coding building blocks prior to, simultaneously with, or following each of the contacting steps.

The compounds of the present invention are prepared using a variety of synthetic reactions, including, but not limited to, amine acylation, reductive alkylation, aromatic reduction, aromatic acylation, aromatic cyclization, aryl-aryl coupling, [3+2] cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2] cycloaddition, enamine formation, esterification, Friedel Crafts reaction, glycosylation, Grignard reaction, Horner-Emmons reaction, hydrolysis, imine formation, metathesis reaction, nucleophilic substitution, oxidation, Pictet-Spengler reaction, Sonogashira reaction, thiazolidine formation, thiourea formation and urea formation. The reactive components of the present invention are those that enable the reactions above to occur. These include, but are not limited to, nucleophiles, electrophiles, acylating agents, aldehydes, carboxylic acids, alcohols, nitro, amino, carboxyl, aryl, heteroaryl, heterocyclyl, boronic acids, phosphorous ylides, etc. In order to encode each scaffold building block, the corresponding coding building block can be prepared by a coding reaction that encodes the functionality of the corresponding scaffold building block. One of skill in the art can envision other synthetic reactions and reactive components useful in the present invention. Table 1 highlights several reactions that can be used to prepare the compounds of the present invention, and the corresponding coding reactions and reactive components. In Table 1, one of skill in the art will understand that radicals R, R¹ and R² can be, for example, hydrogen, alkyl, cycloalkyl, heterocyclyl, aryl and heteroaryl, all optionally substituted. One of skill in the art will further understand that radical Ar is an aryl, which can be, for example, phenyl, naphthyl, pyridyl and thienyl. In addition, one of skill in the art will understand that radical X can be, for example, hydrogen, halogen alkyl, cycloalkyl, heterocyclyl, aryl and heteroaryl.

TABLE 1
Proposed coding strategy for 15 coupling reactions.

| Reactions | Reaction schemes | Reference | Proposed coding reactions |
|---|---|---|---|
| Amine acylation | 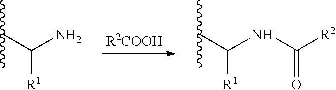 | Perumattam et al. 1998 | 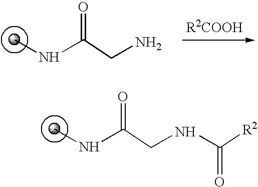 |
| Reductive alkylation | 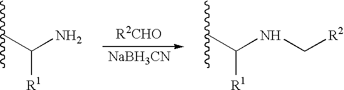 | Gordan and Steele 1995 | 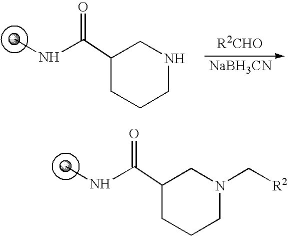 |
| Aromatic reduction, aromatic acylation, aromatic cyclization | 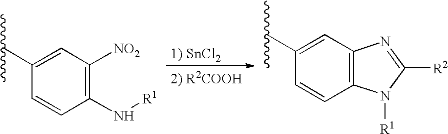 | Mazurov 2000 | 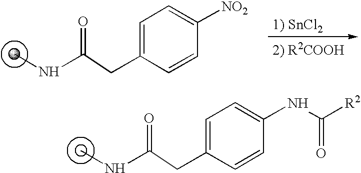 |
| Aryl-Aryl coupling | 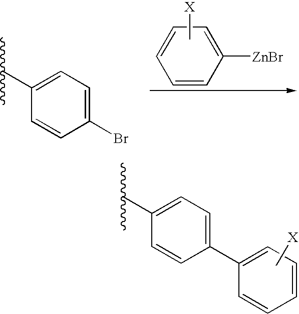 | Marquis and Arlt 1996 | 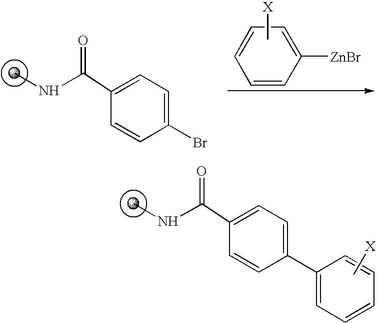 |
| [3 + 2]Cycloaddition | 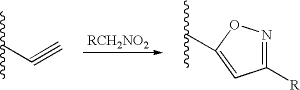 | Park and Kurth 1999 | 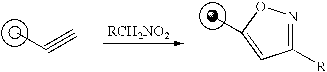 |
| Mitsunobu reaction | 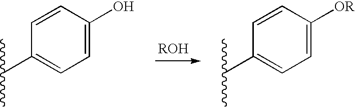 | Gentiles et al. 2002 | 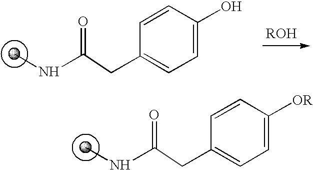 |
| Nucleophilic aromatic substitution | 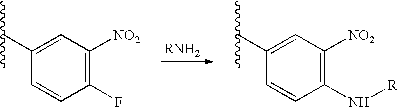 | Wei and Phillips 1998 | 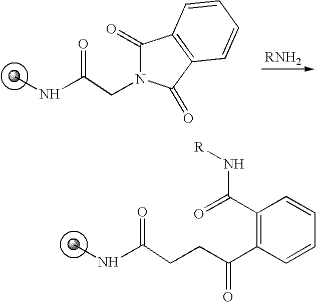 |
| Michael addition | 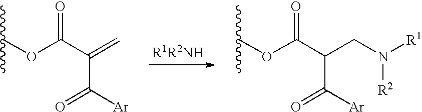 | Garibay et al. 1998 | 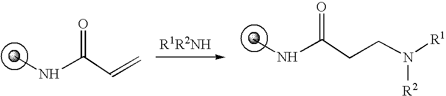 |
| Wittig reaction | 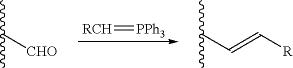 | Veerman et al. 1998 | 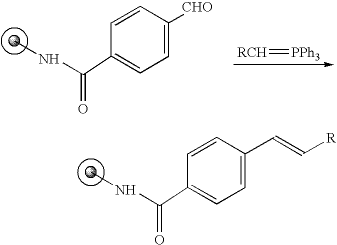 |
| Knoevenagel condensation | 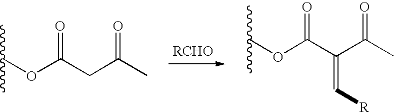 | Gordeev et al. 1996 | 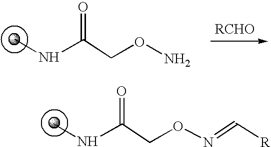 |
| Reductive animation | 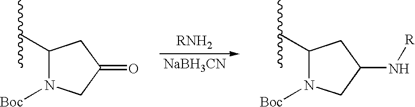 | Bray et al. 1995 | 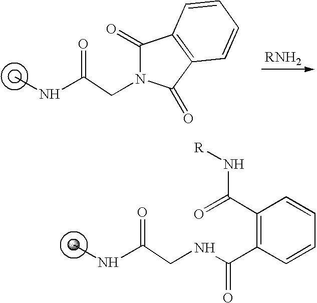 |
| Heck reaction | 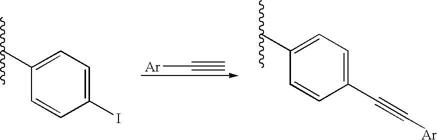 | Yu et al. 1994 | 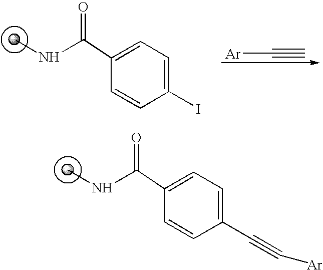 |
| Stille reaction | 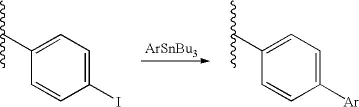 | Forman and Sucholeiki 1995 | 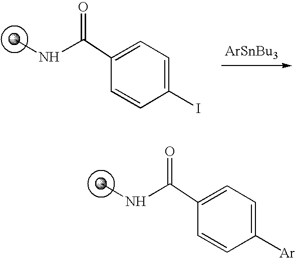 |
| Suzuki reaction | 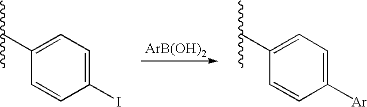 | Frenette and Friesen 1994 | 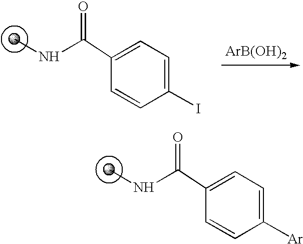 |
| Aldol condensation | 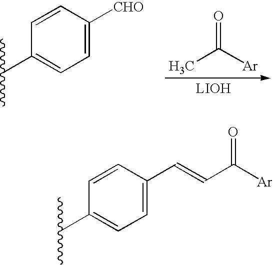 | Marzinzik and Felder 1998 | 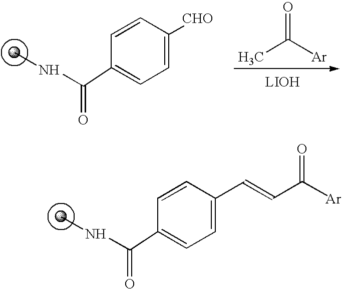 |
| Claisen condensation | 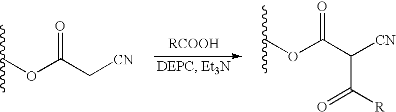 | Sim et al. 1998 | 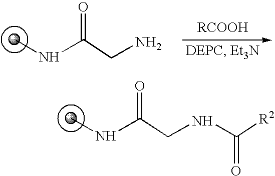 |

Contacting the scaffold functional group with a reactive component results in conversion of the scaffold functional group to the scaffold building block. In a similar manner, contacting the scaffold functional group with ante reactive component results in conversion of the corresponding coding functional group to the appropriate coding building block. In this manner, the scaffold building block is encoded by a coding building block. It would be apparent to one of skill in the art that “contacting” one component with another means to bring them into such close proximity that they can react with one another to afford a third component, the product.

In a preferred embodiment, the compounds of the library are prepared in parallel. In this embodiment, the compounds of the library can be prepared either using the split-mix methodology or in multi-partition containers. One of skill in the art will appreciate that other methods of preparing the compounds of the library in a parallel fashion are useful.

In one embodiment, the present invention provides bead aggregates that comprise units of formula I:

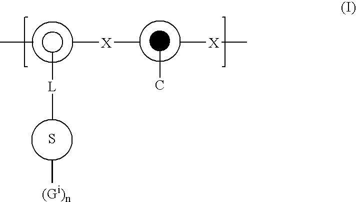

wherein (Gⁱ)ₙ represents n independent scaffold functional groups, G¹ to Gⁿ, wherein each Gⁱ is a scaffold functional group;

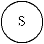

is a scaffold; L is a scaffold linker;

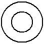

is the compound bead, wherein the inner circle represents an interior portion of the compound bead, and the outer circle represents an exterior portion of the compound bead;

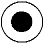

is the coding bead, wherein the darkened portion represents an interior portion of the coding bead, and the lightened portion represents an exterior portion of the coding bead; C represents the coding functional group; X is a crosslinker linking the compound bead to the coding bead; subscript n is an integer from 2 to 10; and superscript i is an integer from 1 to n. In a preferred embodiment, crosslinker X also links together compound beads of the present invention.

In a preferred embodiment, the bead aggregates comprise units of formula Ia:

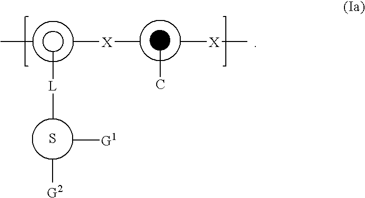

In formula Ia, n=2, resulting in two scaffold functional groups, G¹ and G², each separately attached to the scaffold.

In another embodiment, the bead aggregates comprise units of formula Ib:

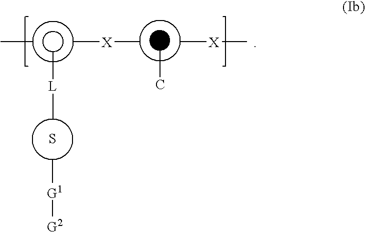

In formula Ib, n=2, resulting in two scaffold functional groups, G¹ and G², wherein G² is linked to the scaffold via G¹.

In yet another embodiment, the bead aggregates comprise units of formula Ic:

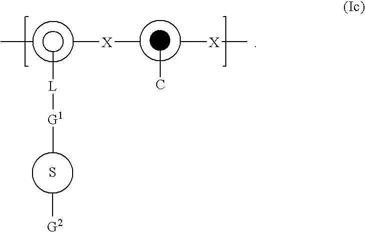

In formula Ic, n=2, resulting in two scaffold functional groups, G¹ and G2, wherein the scaffold is linked to the scaffold linker via G¹.

In a preferred embodiment, steps b)-d) of the method of the present invention afford bead aggregates comprised of units of formula II:

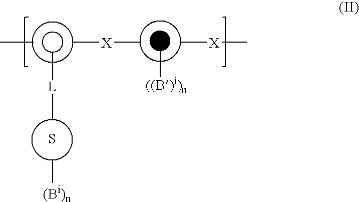

wherein (B¹), represents n independent scaffold building blocks, B¹ to Bⁿ, wherein each Bⁱ is a scaffold building block;

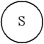

is a scaffold; L is a scaffold linker;

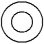

is the compound bead, wherein the inner circle represents an interior portion of the compound bead, and the outer circle represents an exterior portion of the compound bead;

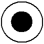

is the coding bead, wherein the darkened portion represents an interior portion of the coding bead, and the lightened portion represents an exterior portion of the coding bead; X is a crosslinker linking the compound bead to the coding bead; subscript n is an integer from 2 to 10; and superscript i is an integer from 1 to n.

Linear Encoding Method

In a preferred embodiment of the present invention, the libraries of the invention are encoded libraries in which the coding sequence on each support corresponds to the structure of the synthetic test compound on each bead aggregate. Thus, each unique synthetic test compound of the library is encoded by a unique coding sequence. Preferably, the coding sequence is a peptide, although the present invention encompasses the use of nucleic acids or any sequenceable polymer as a coding sequence.

For example, the coding sequence may be a peptide. In this case, codes consisting of one or more α-amino acid residues which can be readily detected by Edman degradation, are known to couple efficiently in solid phase peptide synthesis, and where any existing side-chain protecting groups are stable to all the chemistries used in the preparation of the library, are considered to be especially useful.

It is also particularly useful to use α-amino acid residues that do not require side-chain protecting groups. These include, but are not limited to, isoleucine, valine, cyclohexyl-L-alanine, norleucine, norvaline, proline, and the like. Less preferred are asparagine and glutamine. In another embodiment, each of the 20 natural amino acids can code for a specific subunit. A single coding sequence subunit or codon can code for more than one subunit of the synthetic test compound, resulting in a degenerate code, although this is not necessary. One of skill in the art will recognize that non-natural amino acids are also useful as coding building blocks in the coding sequences of the present invention.

An important synthetic operation during the synthesis of an encoded library involves the use of orthogonal protecting groups. For the efficient synthesis of the coding building blocks in parallel with the synthesis of the synthetic test compound of the library on the same solid support particle, the protecting groups used for each synthesis must be orthogonal, i.e., during all synthetic operations on one molecule the protecting groups on the other molecule must remain intact.

Several orthogonal combinations of protecting groups for the assembly of the synthetic test compound and coding molecules of a molecular library can be used. Useful protecting groups are described in Geiger and Konig, 1981, “The Peptides” (Gross and Meinhofer, eds.) pp. 3-101, Academic Press: New York). A very useful combination involves base- and acid-cleavable protecting groups. Many protecting groups useful in the present invention can be found in “Protective Groups in Organic Chemistry”, 3^{rd} ed., T. W. Greene and P. G. M. Wuts, John Wiley & Sons, New York, N.Y., 1999. Other protecting groups useful in the present invention are known to one of skill in the art.

An alternative combination of orthogonal protecting groups in the synthesis of an encoded library of polyamides involves use of Fmoc or other base-labile groups to assemble the coding sequences and Ddz or other acid-labile groups to assemble the ligand binding compounds.

An additional useful combination of orthogonal protecting groups involves the trimethylsilylethoxycarbonyl group, which can be removed by fluoride ions, and a highly acid-sensitive protecting group such as Ddz or Bpoc (2-Biphenyl-2-propoxycarbonyl).

For the synthesis of the peptide coding sequences in preferred encoded libraries, the well-known techniques of solid phase peptide synthesis including suitable protecting group strategies will be used. The relevant published art of peptide synthesis is quite extensive and includes among others Stewart and Young, 1984, “Solid Phase Synthesis”, Second Edition, Pierce Chemical Co., Rockford Ill.; Bodanszky, Y. Klausner, and M. Ondetti, “Peptide Synthesis”, Second Edition, Wiley, N.Y., 1976; E. Gross and J. Meienhofer (editors), “The Peptides”, vol. 1, continuing series, Academic Press, New York, 1979; and “Protective Groups in Organic Chemistry”, 3^{rd} ed., T. W. Greene and P. G. M. Wuts, John Wiley & Sons, New York, N.Y., 1999.

In a preferred embodiment, the encoding step occurs following the contacting step. In another preferred embodiment, subsequent coding building blocks are attached to the coding bead via previously attached coding building blocks. In a more preferred embodiment, the bead aggregates comprise units of formula IIa:

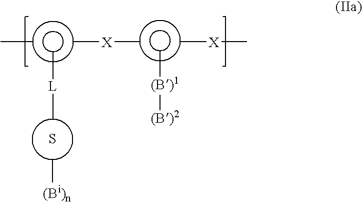

wherein subscript n is 2. In formula Ia, the two coding building blocks ((B′)¹ and (B′)²) are linked to the coding bead in a linear fashion, and together comprise the coding sequence.

Separately-Attached Encoding Method

The encoding strategy of the present invention can also utilize cleavable coding functional groups attached to the coding beads. In one embodiment, the coding functional groups of the present invention include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, amino acid, aryl, cycloalkyl, heterocyclyl, heteroaryl, etc. Each of these coding functional groups is optionally separately linked to the solid support through a coding linker. Each coding functional group that is identical to or mimics a corresponding scaffold functional group on the scaffold of the compound to be synthesized. In a preferred embodiment, the number of the coding functional groups is equal to the number of the scaffold functional groups.

In another preferred embodiment, the encoding step is performed simultaneously with the contacting step. In yet another embodiment, each of the coding building blocks is separately attached to the coding bead. In a further embodiment, the bead aggregates comprise units of formula IIb:

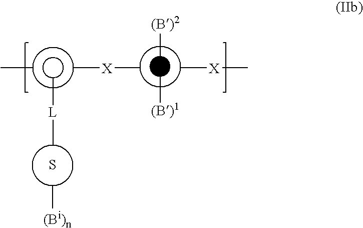

wherein subscript n is 2. In formula Ia, the two coding building blocks ((B′)¹ and (B′)²) are separately linked to the coding bead.

The solid supports of the present invention are first topologically derivatized (vide infra) with a protecting group on the outer layer using a bi-phasic solvent approach (Liu et al. 2002). A cleavable linker, which can facilitate the mass determination of coding building blocks, is then built in the interior of the coding bead. Coding functional groups are chosen according to the scaffold functional groups on the scaffold, and are coupled to the linker. Each coding functional group contains only one functional group, which has the same or similar chemical reactivity as the corresponding scaffold functional group on the scaffold. During the library synthesis, the reactive components couple to the scaffold functional groups and corresponding coding functional groups simultaneously.

Bead Aggregate Library Prepared Using Separately Attached Encoding Methodology. The scaffold, 4, 7-di chloro-2-chloromethyl quinazoline, can be prepared (Scheme 1) using the approach reported by Wright et al. (J Med Chem 2002, 45, 3865-3877).

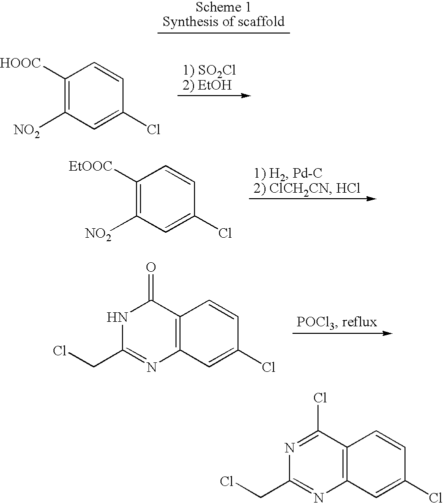

After cleaving the Alloc of the coding linker with Pd(PPh₃)₄/PhSiH₃ in DCM at room temperature for 30 min (twice), the mixture of coding functional group precursors (4-chloromethylbenzoic acid, 4-bromoebenzoic acid, and N-Alloc-nipecotic acid) can be coupled to the coding beads in a pre-determined ratio of reaction activity via HOBt/DIC coupling. (Scheme 2)

After removal of the Fmoc group of both the compound beads and coding beads using 20% piperidine in DMF, the bead aggregates can be split into different portions to which each of the first aldehyde building blocks can be added (one portion receives one aldehyde). The aldehydes react simultaneously, via reductive alkylation, in the compound beads to form secondary amines (first scaffold building block), and in the coding beads with coding functional group nipecotic acid to form tertiary amines (first coding building block).

After the reaction is complete, all the bead aggregates can then be combined and mixed, and then added to a solution of the scaffold. The 4-chloro group of the scaffold is more reactive than the other two chloro groups, and will react first with the secondary amines of the compound beads by nucleophilic substitution.

The bead aggregates can then be split and each portion of bead aggregates receives a second building block (aryl boronic acids). The boronic acids can be coupled to the scaffold and the second coding functional group (4-bromobenzoic acid) simultaneously via Suzuki reaction to prepare the second scaffold building block and the second coding building block.

After another round of mix and split, the third building block (amines) can be coupled to the scaffold and the third coding functional group (chloromethyl benzoic acid) at the same time to prepare the third scaffold building block and the third coding building block. In the last step, high temperature or microwave could be required.

After the synthesis is complete, the bead aggregates can be washed with DCM and compounds cleaved from the compound beads with TFA, and the coding building blocks cleaved and analyzed to decode the compound. In the following Scheme 2, one of skill in the art will understand that radicals R₁ and R₂ can be, for example, hydrogen, alkyl, cycloalkyl, heterocyclyl, aryl and heteroaryl, all optionally substituted. One of skill in the art will further understand that radical Ar is an aryl, which can be, for example, phenyl, naphthyl, pyridyl and thienyl, and that radical B⁻ is a base, which can be, for example, an amine base, a nucleophilic base or a non-nucleophilic base.

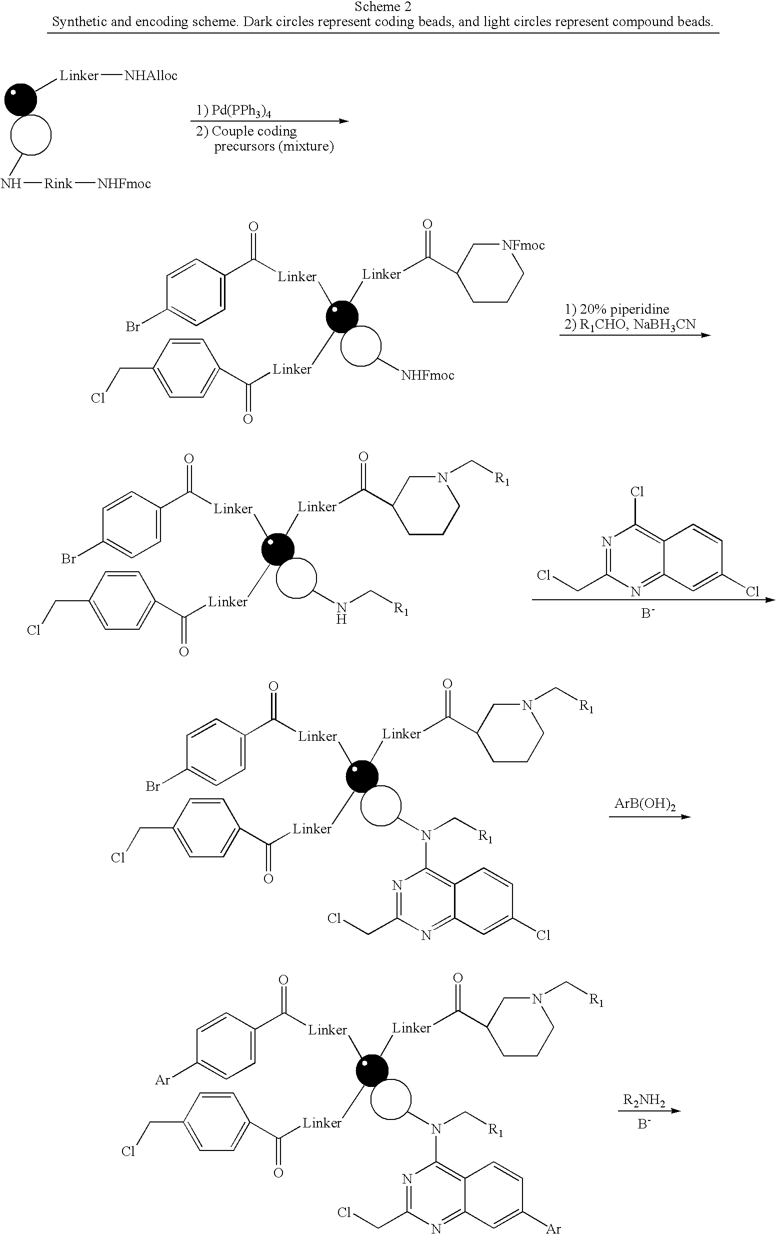

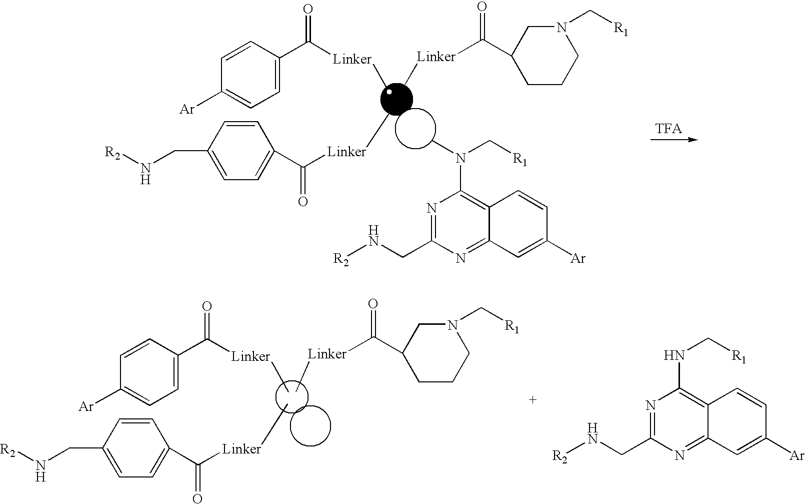

B. Decoding the Library

There are two general approaches to determining the structure of a test compound: the structure of the compound may be directly analyzed by conventional techniques, e.g., nuclear magnetic resonance or mass spectrometry; alternatively, a second molecule or group of molecules can be synthesized during the construction of the library such that the structure(s) of the second molecular species unambiguously indicates (encodes) the structure of the test compound attached to the same support. By this second technique, the structure of compounds that are not themselves amenable to analyzing can be readily determined.

Yet another embodiment of the present invention encompasses a third coding technique, termed “fractional coding,” which differs from the previous embodiments in that there is not a distinct coding molecule different from the test compound. Fractional coding is used when specific subunits of the test compound are not resolvable in conventional analysis, e.g., the D and L stereo isomers of an amino acid. Fractional coding provides a method whereby the subunits can be distinguished by mixing a small amount of a different subunit, not otherwise utilized in the construction of the library, at the time the library is synthesized. Thus, fractional coding creates a minor, detectable degree of heterogeneity of the test compound of the support when one of the two indistinguishable subunits is used. For the purposes of the present invention such a degree of heterogeneity, typically about 5%, is compatible with the teaching of the application that there be only one species of test compound on each support.

In a preferred embodiment of the encoded molecular libraries, the bead aggregate containing the synthetic test compound of interest also contains a coding sequence, preferably a peptide, whose sequence encodes the structure of the ligand, e.g., determination of the sequence of the coding peptide reveals the identity of the ligand. A preferred method of determining the peptide sequencing is Edman degradation. The amino acid sequence of peptides can also be determined either by fast atom bombardment mass spectroscopy (FAB-MS) or using other analytical techniques known to one of skill in the art.

The coding sequences can be sequenced either attached to or cleaved from the solid support. To cleave the coding sequences, the isolated coding beads are treated with traditional cleaving agents known to those of skill in the art to separate peptides from solid phase supports. The choice of cleaving agent selected will depend on the solid phase support employed.

Alternatively, in another embodiment within the scope of the invention, it is possible to isolate a single solid phase support particle, such as a bead, with its coding sequence attached and introduce the bead to a sequencer for peptide sequencing without previously cleaving the coding peptide from the bead. It is estimated that a single 100 μm diameter resin bead with 0.5 mEq/gram of functionalizable sites contains approximately 50 pmole of peptide if one half of the sites are used to link coding peptides. For a similar degree of substitution with coding peptides, a single 250 μm diameter PAM resin bead with 0.5 mEq/gram resin of functionalizable sites contains approximately 1500 μmole of coding peptide. With a state of the art peptide sequencer, only 5-10 pmole is required for adequate sequencing. Therefore, for a standard PAM resin a single bead of 100 μm in diameter can be loaded to contain more than an adequate amount of coding peptide for sequencing.

In addition to Edman sequencing, fast ion bombardment mass spectrometry is a very powerful analytical tool and can often be used effectively to analyze the structures of peptides and of a variety of other molecules. Electrospray-high performance mass spectrometry can also be very useful in structural analysis. Preferably, mass spectrometry to determine the structure of a coding molecule is performed as described in U.S. patent application Ser. No. 07/939,811, filed Sep. 3, 1992.

Those skilled in the art will appreciate that at times the number of species of subunits at any position of the test compound is larger than the number of monomers used to construct the coding sequence. For example, a coding sequence can be constructed with a limited set of amino acids that are readily distinguished after Edman degradation. Under these circumstances the coding sequence can be constructed by introducing a mixture of amino acids at a given position. For example a singlet/doublet code, i.e., having one or two coding moieties per position of the test compound, in which the coding sequence contains only 8 amino acids can encode up to 36 subunits; a triplet/doublet/singlet code with the same number of moieties encodes 84 subunits per position.

The analysis of the Edman degradation products of such coding peptides will reveal either one or two, or one, two or three amino acids at each position of the coding sequence.

Alternatively, decoding can be accomplished by cleaving the coding building blocks and analyzing the releasates by mass spectrometry. In a preferred embodiment, matrix-assisted laser desorption/ionization Fourier transform mass spectrometry (MALDI-FTMS) is used due to its high mass resolution, accuracy and sensitivity. A hydrophilic linker (-linker-Phe-Phe-Met-) that links the coding molecules with solid support (resin bead) is designed to facilitate mass spectrometry analysis. Methionine is stable to many chemical reactions, but it can be readily cleaved by cyanogen bromide (CNBr). Its cleavage is very reliable and specific, and offers clean products, which are suitable to single-bead analysis. Two phenylalanines are introduced into the linker to increase the molecular weight of the final cleavage products, so that their signals can be easily distinguished from those of matrix and impurities. An additional hydrophilic linker is selected to enhance the solubility of final cleaved products in extraction solvent (50% acetonitrile/water). The whole linker has excellent chemical stability, and is very suitable for MALDI-FTMS detection.

Using this method, it is possible to detect several coding building blocks of a single bead. Because only the molecular mass of coding building blocks is needed to identify the structure of library compound, a very small amount of coding building blocks is enough for MALDI-FTMS detection. Considering a library based on a scaffold with four diversities, if 100 different reactive components are used in each synthetic step, a library containing 100⁴=100,000,000 compounds will be generated, while the total number of coding building block structures required is only 400. Because of the high precision and sensitivity of MALDI-FTMS, it is not difficult to accurately identify each of the 400 different building blocks used in the library synthesis. Since each coding functional group has only one functional group, the chemical structure of final coding building blocks is very simple.

C. Solid Supports

A separate phase support suitable for use in the present invention is characterized by the following properties: (1) insolubility in liquid phases used for synthesis or screening; (2) capable of mobility in three dimensions independent of all other supports; (3) containing many copies of each of the synthetic test compound and, if present, the coding sequence attached to the support; (4) compatibility with screening assay conditions; and (5) being inert to the reaction conditions for synthesis of a test compound. A preferred support also has reactive functional groups, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching a subunit which is a precursor to each of the synthetic test compound and coding building blocks, or for attaching a linker which contains one or more reactive groups for the attachment of the monomer or other subunit precursor.

As used herein, separate phase support is not limited to a specific type of support. Rather a large number of supports are available and are known to one of ordinary skill in the art. In a preferred aspect, the separate phase support is a solid phase support, although the present invention encompasses the use of semi-solids, such as aerogels and hydrogels. Solid phase supports include silica gels, resins, derivatized plastic films, glass beads, cotton, plastic beads, alumina gels, polysaccharides such as Sepharose and the like, etc. A suitable solid phase support can be selected on the basis of desired end use and suitability for various synthetic protocols. For example, in polyamide synthesis, useful solid phase support can be resins such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany) or polydimethyl-acrylamide resin (available from Milligen/Biosearch, California). Preferred solid phase synthesis supports for specific syntheses are described below. Thus, each resin bead is functionalized to contain both synthetic test compound and the corresponding coding structures. In a variation of this approach, the synthetic test compound and coding building blocks are attached to the solid support through linkers such as those described below. One of skill in the art will recognize that while many types of solid supports are useful in the present invention, topologically segregated solid supports are particularly useful.

Topology of Solid Supports

A variety of approaches for topologically separating the synthetic test compound and coding building blocks on a solid support in order to generate libraries are useful.

Topologically separating the synthetic test compound and the coding building block refers to the separation in space on a support. For example, if the support is a resin bead, separation can be between the surface and the interior of the resin bead of a significant number of the ligand-candidate molecules from a significant number of the coding building blocks. Preferably, the surface of the support contains primarily synthetic test compound molecules and very few coding building blocks. More preferably, the surface of the support contains greater than 90% synthetic test compound and less than 10% coding building blocks. Even more preferably, the surface of the support contains greater than 99% synthetic test compound molecules and less than 1% coding building blocks; most preferably, it contains more than 99.9% synthetic test compound and less than 0.1% coding building blocks. The advantage of such an arrangement is that interference of the coding building block in a binding screening assay is limited. It is not necessary that the topological area that contains the coding sequence, i.e., the interior of a resin bead, be free of the synthetic test compound.

As discussed above, the coding building blocks are optionally segregated in the interior of the support particle. However, coding building blocks can also be segregated to the surface of a support particle, or to one side of a support particle.

One general approach for the topological separation of synthetic test compound from coding building blocks involves the selective derivatization of reactive sites on the support based on the differential accessibility of the coupling sites to reagents and solvents. For example, regions of low accessibility in a resin bead are the interior of the bead, e.g., various channels and other cavities. The surface of a resin bead, which is in contact with the molecules of the solution in which the bead is suspended, is a region of relatively high accessibility. Methods for effecting the selective linkage of coding functional groups and scaffolds to a suitable solid phase support include, but are not limited to, the following.

(i) Selective Derivatization of Solid Support Surfaces Via Controlled Photolysis

Two approaches can be used. In one, a functionalized solid support is protected with a photocleavable protecting group, e.g., nitroveratryloxycarbonyl (Nvoc) (Patchornik et al. J. Am. Chem. Soc. 1970, 92, 6333). The Nvoc-derivatized support particles are arranged in a monolayer formation on a suitable surface. The monolayer is photolyzed using light of controlled intensity so that the area of the bead most likely to be deprotected by light will be the area of the bead in most direct contact with the light, i.e., the exterior surface of the bead. The resulting partially deprotected beads are washed thoroughly and reacted with a scaffold containing a light-stable protecting group. Following the reaction with the scaffold, the beads are subjected to quantitative photolysis to remove the remaining light-sensitive protecting groups, thus exposing functional groups in less light-accessible environments, e.g., the interior of a resin bead. After this quantitative photolysis, the support particles are further derivatized with an orthogonally-protected coding functional group, e.g., Fmoc-protected amino acid. The resulting solid support bead will ultimately contain synthetic test compound segregated primarily on the exterior surface and coding building blocks located in the interior of the solid phase support bead (see Scheme 1).

An alternative photolytic technique for segregating coding building blocks and synthetic test compound on a support involves derivatizing the support with a branched linker, one branch of which is photocleavable, and attaching the coding functional groups to the photosensitive branch of the linker. After completion of the synthesis, the support beads are arranged in a monolayer formation and photolyzed as described above. This photolysis provides beads which contain patches of synthetic test compound for selective screening with minimal interference from the coding building blocks.

(ii) Selective Derivatization of Solid Support Surfaces Using Chemical or Biochemical Approaches

The efficacy of these chemical and biochemical derivatizations depends on the ability of exterior surface functional groups, which are exposed, to react faster than other groups in the interior which are not exposed. It has been observed, for example, that antibodies cannot bind to peptide ligands in the interior of a resin solid phase support. Therefore, using differences in steric hindrance imposed by the structure of the support or by modulating the swelling of a bead through choice of reaction solvent, reactive groups on the exterior of the bead that are accessible to macromolecules or certain reagents can be reacted selectively relative to reactive groups in the interior of the bead. Therefore, the reactive groups in the exterior of the bead can be modified for the synthesis of the synthetic test compound, while interior reactive groups can be modified for preparation of the coding building blocks, or both the coding building blocks and synthetic test compound. Since the number of reactive groups inside a resin bead is much larger than the number of groups on the outer surface, the actual number of coding building blocks will be very large, providing enough coding building blocks for accurate mass spectral analysis, and thus the decoding of the structure of the synthetic test compound. A variety of chemical and biochemical approaches are contemplated including the following:

(a) Use of Polymeric Deprotecting Agents to Selectively Deprotect Parts of the Exterior of a Solid Support Bead Carrying Protected Functional Groups

The deprotected functional groups are used as anchors for the scaffold. The functional groups which remain protected are subsequently deprotected using a nonpolymeric deprotecting agent and used as anchors for the attachment of the coding functional groups. In a specific embodiment, this method involves use of enzymes to selectively activate groups located on the exterior of beads which have been derivatized with a suitable enzyme substrate. Due to their size, enzymes are excluded from the interior of the bead. In an example, infra, an enzyme completely removes a substrate from the surface of a resin bead, without significantly affecting the total amount of substrate attached to the bead, i.e., the interior of the bead. The removal of substrate exposes, and thus activates, a reactive site on the bead. The enzyme-modified groups of the solid support are used to anchor the scaffold and those groups that escaped modification are used to anchor the majority of the coding functional groups.

(b) Use of a Polymeric Protecting Group to Selectively Building Block Exposed Unprotected Functional Groups on the Exterior of a Support Bead

The unprotected functional groups in the interior of the support are used to anchor the coding functional groups. The remaining protected functional groups are then deprotected and used as anchors for the scaffolds of the library.

(c) Creating a Different State in the Interior of the Bead

Through the judicious selection of solvents, it is possible to swell the beads with one solvent, which is subsequently frozen, and then add the beads to a second solvent at a low temperature. For example, by freezing water inside the beads, then reacting the beads in an organic solvent at low temperature, the water in the interior of the bead remains frozen. Thus the surface of the bead, but not the interior, can be selectively reacted.

(d) Use of a Biphasic Solvent Environment

In a similar fashion to method (c) above, the beads are first swelled with an aqueous solvent, followed by derivatization of the beads in an appropriate organic solvent such that the water in the interior of the bead remains there. In this manner, only the functional groups on the outside of the bead (those not in the aqueous solvent) are derivatized (Liu, R. et al. J. of the Am. Chem. Soc. 2002, 124, 7678).

Bead Aggregates

The bead aggregates of the present invention are preferably prepared following the procedure in Example 1. One of skill in the art can envision other useful methods of preparing the bead aggregates of the present invention.

The solid supports of the present invention can further comprise grafted polymer chains attached to the exterior of the beads. In another embodiment, the grafted polymer chains can be attached to the interior. The grafted polymer chains preferably contain amino functionalities similar to those on the beads. Upon crosslinking the beads of the present invention, the amino functionalities on the grafted polymer chains will also react and further crosslink the beads. One of skill in the art can envision other chemical functionalities on the grafted polymer chains that would also lead to an increase in the crosslinking.

In one embodiment, the crosslinked grafted polymer chains improve the stability of the bead aggregates by increasing the number of crosslinks between the individual beads. The grafted polymer chains can be prepared by attaching polymer initiators to the exterior of the beads, and a copolymer of OEGMAm and a Boc-protected, amine containing acrylamide monomer can then be grafted to the surface of these beads. The Boc groups can be removed using standard TFA treatment. One of skill in the art can envision other homopolymers and copolymers that are useful in the present invention.

In a preferred embodiment, the compound beads and the coding beads are present in each of the bead aggregates in a ratio of 99.9/0.1 to 50.0/50.0. In a more preferred embodiment the compound beads and the coding beads are present in a ratio of 99/1 to 90/10. In a most preferred embodiment, the compound beads and the coding beads are present in a ratio of 98/2 to 95/5.

D. Linkers

The solid supports of the present invention can also comprise linkers or an arrangement of linkers. As used herein, a linker refers to any molecule containing a chain of atoms, e.g., carbon, nitrogen, oxygen, sulfur, etc., that serves to link the molecules to be synthesized on the solid support with the solid support. The linker is usually attached to the support via a covalent bond, before synthesis on the support starts, and provides one or more sites for attachment of precursors of the molecules to be synthesized on the solid support. Various linkers can be used to attach the precursors of molecules to be synthesized to the solid phase support. Examples of linkers include aminobutyric acid, aminocaproic acid, 7-aminoheptanoic acid, 8-aminocaprylic acid, lysine, iminodiacetic acid, polyoxyethylene, glutamic acid, etc. In a further embodiment, linkers can additionally comprise one or more alanines or other amino acids as spacers.

In another embodiment, the “safety-catch amide linker” (SCAL) (see Patek, M. and Lebl, M. 1991, Tetrahedron Letters 1991, 32, 389 1; International Patent Publication WO 92/18144, published Oct. 29, 1992) is introduced to the solid support.

In addition to the linkers described above, selectively cleavable linkers can be employed. One example is the ultraviolet light sensitive linker, ONb, described by Barany and Albericio (J. Am. Chem. Soc. 1985, 107, 4936). Other examples of photocleavable linkers are found in Wang (J.Org. Chem. 1976, 41, 32), Hammer et al (Int. J Pept. Protein Res. 1990, 36, 31), and Kreib-Cordonier et al. in “Peptides—Chemistry, Structure and Biology”, Rivier and Marshall, eds., 1990, pp. 895-897). Landen (Methods Enzym. 1977, 47, 145) used aqueous formic acid to cleave Asp-Pro bonds; this approach has been used to characterize T-cell determinants in conjunction with the Geysen pin synthesis method (Van der Zee et al. 1989, Eur. J. Immunol. 191: 43-47). Other potential linkers cleavable under basic conditions include those based on p-(hydroxymethyl)benzoic acid (Atherton et al. 1981, J. Chem. Soc. Perkin I: 538-546) and hydroxyacetic acid (Baleaux et al. 1986, Int. J. Pept. Protein Res. 28: 22-28). Geysen et al. (1990, J. Immunol. Methods 134: 23-33; International Publication WO 90/09395) reported peptide cleavage by a diketopiperazine mechanism. Preferred diketopiperazine linkages are disclosed in U.S. Pat. No. 5,504,265, which is hereby incorporated by reference in its entirety.

Enzyme-cleavable linkers can also be useful. An enzyme can specifically cleave a linker that comprises a sequence that is recognized by the enzyme. Thus, linkers containing suitable peptide sequences can be cleaved by a protease and linkers containing suitable nucleotide sequences can be cleaved by an endonuclease.

In certain instances, one can derivatize a portion (e.g., 10-90%) of the available resin functional groups with a cleavable linker using certain reaction conditions, and the remaining of the resin functional groups with a linker which is stable to the cleavage conditions to ensure that enough material will remain on the resin after cleavage for further study. This arrangement is particularly preferred when there are no coding molecules. Combinations of linkers cleavable under different reaction conditions can also be used to allow selective cleavage of molecules from a single solid support bead.

A solid phase support linker for use in the present invention can further comprise a molecule of interest, which can be further derivatized to give a molecular library. The pre-attached molecule can be selected according to the methods described herein, or can comprise a structure known to embody desired properties. In a preferred embodiment, the scaffold linker is an amino acid.

An ionization linker has been used to enhance ionization of poorly- or non-ionizablemolecules (Carrasco, M. R., et al. Tetrahedron Lett. 1997, 38, 6331-6334). The linker also provides a mass shift which overcomes signal overlap with matrix molecules. To effectively decode each bead with mass spectrometry, the linker should meet the following four criteria. First, the linker must be inert to the chemical reactions for library synthesis and stable under the conditions used for various biological screening. Second, the linker should be highly sensitive to the ionization method so that the final coding building blocks with different structures can be readily detected. Third, its cleavage must be clean and efficient. Fourth, the linker should have excellent solubility in the extraction solvent. A simple peptide-like linker that meets the above four criteria has been designed and synthesized on solid phase using the standard Fmoc chemistry (Fields, G. B., et al. Int. J. Peptide Protein Res. 1990, 35, 161-214). In principle, any chemically cleavable or photosensitive linkers can be used as the cleavable part as long as they are compatible with the library synthesis and screening. Methionine is preferred due to its clean and specific cleavage by cyanogen bromide (CNBr), and the final homoserine lactone product (Gross, E. et al. J. Biol. Chem. 1962, 237, 1856-1860) is chemically stable. This cleavage method has been successfully applied to single-bead analysis of peptides (Youngquist, R. S. et al. Rapid Commun. Mass Spectrom. 1994, 8, 77-81; Youngquist, R. S., et al. J. Am. Chem. Soc. 1995,117,3900-3906). Two phenylalanines are coupled to the methionine to increase the molecular weight of the linker. Finally, a linear hydrophilic molecule is introduced to the linker to enhance solubility of the coding building block in the extraction solvent (50% acetonitrile/water). The whole linker has excellent chemical stability, and is very suitable for MALDI-FTMS detection. The oxygen atoms, the amide bonds and the side chain of phenylalanines in the linker allow efficient formation of primarily sodiated species, and therefore provide efficient ionization.

E. Scaffolds

Scaffolds of the present invention can be a cyclic or bicyclic hydrocarbon, a steroid, a sugar, a heterocyclic structure, a polycyclic aromatic molecule, an amine, an amino acid, a multi-functional small molecule, a peptide or a polymer having various substituents at defined positions. Preferred scaffolds of the present invention include, but are not limited to, quinazoline, tricyclic quinazoline, purine, pyrimidine, phenylamine-pyrimidine, phthalazine, benzylidene malononitrile, amino acid, tertiary amine, peptide, aromatic compounds containing ortho-nitro fluoride(s), aromatic compounds containing para-nitro fluoride(s), aromatic compounds containing ortho-nitro chloromethyl, aromatic compounds containing ortho-nitro bromomethyl, lactam, sultam, lactone, pyrrole, pyrrolidine, pyrrolinone, oxazole, isoxazole, oxazoline, isoxazoline, oxazolinone, isoxazolinone, thiazole, thiazolidinone, hydantoin, pyrazole, pyrazoline, pyrazolone, imidazole, imidazolidine, imidazolone, triazole, thiadiazole, oxadiazole, benzofuran, isobenzofuran, dihydrobenzofuran, dihydroisobenzofuran, indole, indoline, benzoxazole, oxindole, indolizine, benzimidazole, benzimidazolone, pyridine, piperidine, piperidinone, pyrimidinone, piperazine, piperazinone, diketopiperazine, metathiazanone, morpholine, thiomorpholine, phenol, dihydropyran, quinoline, isoquinoline, quinolinone, isoquinolinone, quinolone, quinazolinone, quinoxalinone, benzopiperazinone, quinazolinedione, benzazepine and azepine. Scaffolds of the present invention also comprise at least two scaffold functional groups including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the scaffold building block. One of skill in the art can envision that other scaffolds, such as a single carbon atom or even a bond, are also useful in the present invention.

In a preferred embodiment, the scaffold is the same on each of the synthesis templates. In another preferred embodiment, at least two different scaffolds are used. In yet another preferred embodiment, the scaffold is a member selected from the group consisting of quinazoline, tricyclic quinazoline, purine, pyrimidine, phenylamine-pyrimidine, phthalazine, benzylidene malononitrile, amino acid, tertiary amine, peptide and polymer. Other scaffolds useful in the present invention will be apparent to one of skill in the art.

F. Split-Mix Methodology

In another preferred embodiment, the library of compounds is prepared via a split-mix methodology. In another aspect, the present invention provides a method for preparing a library of compounds via the split-mix methodology, comprising: a) providing a plurality of individual bead aggregates, wherein each of the bead aggregates comprises a population of compound beads and a population of coding beads, wherein the compound beads and the coding beads are crosslinked to each other, wherein each of the compound beads comprises a scaffold linked to the compound bead via a scaffold linker, and with at least two scaffold functional groups attached to the scaffold, and wherein each of the coding beads comprises at least one coding functional group; b) splitting the bead aggregates into two or more separate pools; c) contacting the bead aggregates with one or more first reactive components in the two or more separate pools such that a first scaffold functional group reacts with one of the first reactive components to afford a first scaffold building block, wherein the contacting step affords subsequent bead aggregates; d) encoding each of the scaffold building blocks with a coding building block, comprising the step of contacting the coding functional group with a reactive component such that the coding functional group reacts with the reactive component to afford a coding building block linked to the coding bead, wherein the coding building block encodes one of the scaffold building blocks, and wherein the encoding step yields subsequent encoded bead aggregates; e) mixing the subsequent encoded bead aggregates from the two or more separate pools into a single pool; f) splitting the subsequent encoded bead aggregates into two or more separate pools; g) contacting the subsequent encoded bead aggregates in the two or more separate pools with a successive reactive component such that a subsequent scaffold functional group reacts with the successive reactive component to afford a subsequent scaffold building block, wherein the contacting step yields further bead aggregates; h) repeating step d), wherein the encoding step yields further encoded bead aggregates; and i) repeating steps e)-h), wherein the further encoded bead aggregates of step h) become the subsequent encoded bead aggregates of step e), until the library of compounds has been prepared.

The synthesis of libraries of synthetic test compound via a split-mix methodology comprises repeating the following steps: (i) dividing the selected support into a number of portions which is at least equal to the number of different subunits to be linked; (ii) chemically linking one and only one of the subunits of the synthetic test compound with one and only one of the portions of the solid support from step (i), preferably making certain that the chemical link-forming reaction is driven to completion to the fullest extent possible; (iii) thoroughly mixing the solid support portions containing the growing synthetic test compound; (iv) repeating steps (i) through (iii) a number of times equal to the number of subunits in each of the synthetic test compound of the desired library, thus growing the synthetic test compound; (v) removing any protecting groups that were used during the assembly of the synthetic test compound on the solid support.

Preferably, the coding building blocks are synthesized in parallel with the synthetic test compound. In this instance, before or after linking the subunit of the synthetic test compound to the support in step (ii), one coding building block, that correspond(s) to the added subunit of the synthetic test compound, is separately linked to the solid support, such that a unique structural code, corresponding to the structure of the growing synthetic test compound, is created on each support. It can be readily appreciated that if an encoded library is prepared, synthesis of the coding unit must precede the mixing step, (iii).

The repetition of steps (i)-(iii) (see step (iv)) will naturally result in growing the synthetic test compound and, if the process is modified to include synthesis of coding building blocks, a coding building block in parallel with each step of the test compound.

In one embodiment, enough support particles are used so that there is a high probability that every possible structure of the synthetic test compound is present in the library. Such a library is referred to as a “complete” library. To ensure a high probability of representation of every structure requires use of a number of supports in excess, e.g., by five-fold, twenty-fold, etc., according to statistics, such as Poisson statistics, of the number of possible species of compounds. In another embodiment, especially where the number of possible structures exceeds the number of supports, not every possible structure is represented in the library. Such “incomplete” libraries are also very useful.

IV. Screening Methods

In addition to providing libraries of a great variety of chemical structures as synthetic test compound, and methods of synthesis thereof, the present invention provides a method for identifying a compound of the present invention that binds to a target, wherein the compound is not attached to a solid support, the method comprising: a) contacting the compound according to the method described above with the target; and b) determining the functional effect of the compound upon the target. In a preferred embodiment, the target of the present invention is a biological target. In other embodiments, the target can be synthetic in nature, such as a photogenic receptor or other material with an intensity physical property.

In another preferred embodiment, the present invention provides a method for determining the functional effect on a target of a compound not attached to a solid support, wherein the target is a protein tyrosine kinase. In a more preferred embodiment, the target is a protein tyrosine kinase.

The methods of screening the test compounds of a library of the present invention identify ligands within the library that demonstrate a biological activity of interest, such as binding, stimulation, inhibition, toxicity, taste, etc. Other libraries can be screened according to the methods described infra for enzyme activity, enzyme inhibitory activity, and chemical and physical properties of interest. Many screening assays are well known in the art; numerous screening assays are also described in U.S. Pat. No. 5,650,489.

The ligands discovered during an initial screening may not be the optimal ligands. In fact, it is often preferable to synthesize a second library based on the structures of the ligands selected during the first screening. In this way, one may be able to identify ligands of higher activity.

A. Binding Assays

The present invention allows identification of synthetic test compounds that bind to acceptor molecules. As used herein, the term “acceptor molecule” refers to any molecule which binds to a ligand. Acceptor molecules can be biological macromolecules such as antibodies, receptors, enzymes, nucleic acids, or smaller molecules such as certain carbohydrates, lipids, organic compounds serving as drugs, metals, etc.

The synthetic test compound in libraries of the present invention can potentially interact with many different acceptor molecules. By identifying the particular ligand species to which a specific acceptor molecule binds, it becomes possible to physically isolate the ligand species of interest.

Because only a small number of solid support beads will be removed during each screening/detection/isolation step, the majority of the beads will remain in the bead pool. Therefore, the library can be reused multiple times. If different color or identification schemes are used for different acceptor molecules (e.g., with fluorescent reporting groups such as fluorescein (green), Texas Red (Red), DAPI (blue) and BODIPI tagged on the acceptors), and with suitable excitation filters in the fluorescence microscope or the fluorescence detector, different acceptors (receptors) can be added to a library and evaluated simultaneously to facilitate rapid screening for specific targets. These strategies not only reduce cost, but also increase the number of acceptor molecules that can be screened.

In the method of the present invention, an acceptor molecule of interest is introduced to the library where it will recognize and bind to one or more ligand species within the library. Each ligand species to which the acceptor molecule binds will be found on a single solid phase support so that the support, and thus the ligand, can be readily identified and isolated.

The desired ligand can be isolated by any conventional means known to those of ordinary skill in the art and the present invention is not limited by the method of isolation. For example, and not by way of limitation, it is possible to physically isolate a solid-support-bead ligand combination that exhibits the strongest physico-chemical interaction with the specific acceptor molecule. In one embodiment, a solution of specific acceptor molecules is added to a library which contains 10⁵ to 10⁷ solid phase support beads. The acceptor molecule is incubated with the beads for a time sufficient to allow binding to occur. Thereafter, the complex of the acceptor molecule and the ligand bound to the support bead is isolated. More specific embodiments are set forth in the following methods, which describe the use of a monoclonal antibody, as a soluble acceptor molecule to bind a ligand which is a peptide. It will be clear that these methods are readily adaptable to detect binding of any acceptor molecule.

In addition to using soluble acceptor molecules, in another embodiment, it is possible to detect ligands that bind to cell surface receptors using intact cells. The use of intact cells is preferred for use with receptors that are multi-subunit or labile or with receptors that require the lipid domain of the cell membrane to be functional. The cells used in this technique can be either live or fixed cells. The cells can be incubated with the library and can bind to certain peptides in the library to form a “rosette” between the target cells and the relevant bead-peptide. The rosette can thereafter be isolated by differential centrifugation or removed physically under a dissecting microscope.

Alternatively, one can screen the library using a panning procedure with cell lines such as (i) a “parental” cell line where the receptor of interest is absent on its cell surface; and (ii) a receptor-positive cell line, e.g., a cell line which is derived by transfecting the parental line with the gene coding for the receptor of interest. It is then possible to screen the library by the following strategy: (i) first depleting the library of its non-specific beads that will bind to the cells lacking the receptor by introducing a monolayer of parental cell line by the standard “panning technique” to leave receptor-specific non-binding beads, or irrelevant non-binding beads; (ii) removing the non-binding beads which will include both receptor-specific or irrelevant beads and loading them on a monolayer of receptor positive cell line in which the receptor-specific bead will bind to the receptor positive cell line; (iii) removing the remaining irrelevant non-binding beads by gentle washing and decanting; and (iv) removing the receptor-specific bead(s) with a micromanipulator, such as a micropipette.

As an alternative to whole cell assays for membrane bound receptors or receptors that require the lipid domain of the cell membrane to be functional, the receptor molecules can be reconstituted into liposomes where reporting group or enzyme can be attached.

The foregoing examples refer to synthetic test compound, and any of the compounds described previously, can be used in the practice of the instant invention. Thus, an acceptor molecule can bind to one of a variety of polyamides, polyurethanes, polyesters, polyfunctionalized structure capable of acting as a scaffolding, etc.

In one embodiment, the acceptor molecule can be directly labeled. In another embodiment, a labeled secondary reagent can be used to detect binding of an acceptor molecule to a solid phase support particle containing a ligand of interest. Binding can be detected by in situ formation of a chromophore by an enzyme label. Suitable enzymes include, but are not limited to, alkaline phosphatase and horseradish peroxidase. In a further embodiment, a two color assay, using two chromogenic substrates with two enzyme labels on different acceptor molecules of interest, can be used. Cross-reactive and singly-reactive ligands can be identified with a two-color assay.

Other labels for use in the present invention include colored latex beads, magnetic beads, fluorescent labels (e.g., fluoresceine isothiocyanate (FITC), phycoerythrin (PE), Texas red (TR), rhodamine, free or chelated lanthanide series salts, especially Eu³⁺, to name a few fluorophores), chemiluminescent molecules, radio-isotopes, or magnetic resonance imaging labels. Two color assays can be performed with two or more colored latex beads, or fluorophores that emit at different wavelengths. Labeled beads can be isolated manually or by mechanical means. Mechanical means include fluorescence activated sorting, i.e., analogous to FACS, and micromanipulator removal means.

In specific examples, enzyme-chromogen labels and fluorescent (FITC) labels are used.

Reactive beads can be isolated on the basis of intensity of label, e.g., color intensity, fluorescence intensity, magnetic strength, or radioactivity, to mention a few criteria. The most intensely labeled beads can be selected and the ligand attached to the bead can be structurally characterized directly e.g., by Edman sequencing or by mass spectral analysis if applicable, or indirectly by sequencing the coding peptide corresponding to the ligand of interest. In another embodiment, a random selection of beads with a label intensity above an arbitrary cut-off can be selected and subjected to structural analysis. One can potentially use modem image analysis microscopy to quantitate the color intensity, and hence precisely define the relative affinity of the ligand to the acceptor molecule prior to the structure analysis of the bead ligand. Similarly, quantitative immunofluorescence microscopy can be applied if the acceptor is tagged with a fluorescent label. In yet another embodiment, beads demonstrating a certain label intensity are selected for compositional analysis, e.g., amino acid composition analysis in the case of peptide ligands. A refinement library comprising a restricted set of amino acid subunits identified as important from the amino acid analysis can then be prepared and screened.

In another embodiment, the ligand(s) with the greatest binding affinity can be identified by progressively diluting the acceptor molecule of interest until binding to only a few solid phase support beads of the library is detected. Alternatively, stringency of the binding with the acceptor molecule, can be increased. One of ordinary skill would understand that stringency of binding can be increased by (i) increasing solution ionic strength; (ii) increasing the concentration of denaturing compounds such as urea; (iii) increasing or decreasing assay solution pH; (iv) use of a monovalent acceptor molecule; (v) inclusion of a defined concentration of known competitor into the reaction mixture; and (vi) lowering the acceptor concentration. Other means of changing solution components to change binding interactions are well known in the art.

In another embodiment, ligands that demonstrate low affinity binding may be of interest. These can be selected by first removing all high affinity ligands and then detecting binding under low stringency or less dilute conditions.

In a preferred embodiment, a dual label assay can be used. The first label can be used to detect non-specific binding of an acceptor molecule of interest to beads in the presence of soluble ligand. Labeled beads are then removed from the library, and the soluble ligand is removed. Then specific binding acceptor molecule to the remaining beads is detected. Ligands on such beads can be expected to bind the acceptor molecule at the same binding site as the ligand of interest, and thus to mimic the ligand of interest. The dual label assay provides the advantage that the acceptor molecule of interest need not be purified since the first step of the assay allows removal of non-specific positive reacting beads. In a preferred embodiment, fluorescent-labeled acceptor molecules can be used as a probe to screen a synthetic test library, e.g., using FACS.

B. Bioactivity Assays

The instant invention further provides assays for biological activity of a ligand-candidate from a library treated so as to remove any toxic molecules remaining from synthesis, e.g., by neutralization and extensive washing of the bead-aggregate library prior to cleavage, with solvent, sterile water and culture medium. The biological activities of the releasates that can be assayed include toxicity and killing, stimulation and growth promotion, signal transduction, biochemical and biophysical changes, physiological change, and enzyme inhibition.

In a preferred embodiment, the synthetic test compounds of the library are selectively cleavable from the solid-phase support, also referred to herein as “bead”. Preferably, the synthetic test compounds are attached to the separate phase support via multiple cleavable linkers to allow for more than one release and screening assay. In one embodiment, beads are prepared such that only a fraction of synthetic test compound are selectively cleavable. A library is treated with a cleaving agent such that cleavage of a fraction of synthetic test compound occurs. Examples of cleaving agents include, but are not limited to, UV light, acid, base, enzyme, or catalyst. In one embodiment, the library is treated so that 10-90% of the synthetic test compound are released. In a more preferred embodiment, 25-50% of the synthetic test compound are released. Where all synthetic test compound molecules are cleavable, non-quantitative cleavage can be effected by limiting the cleaving agent. In one aspect, exposure time and intensity of UV light is limited. In another embodiment, the concentration of reagent is limited. After treatment to effect cleavage, the library can be further treated, e.g., by neutralization, to make it biologically compatible with the desired assay. In practice, one of ordinary skill would be able to readily determine appropriate cleavage conditions for partial cleavage when all synthetic test compound molecules of the library are attached to solid phase by cleavable linkers or bonds. One of ordinary skill would further understand that the relative concentration of released synthetic test compound can be affected by varying the cleavage conditions.

In another preferred embodiment, all the synthetic test compounds of the library are cleavable from the solid-phase support. In a more preferred embodiment, all the synthetic test compounds are cleaved and collected, followed by analysis using conventional screening assays.

It will further be understood by one of ordinary skill in the art that any cell that can be maintained in tissue culture, either for a short or long term, can be used in a biological assay. The term “cell” as used here is intended to include prokaryotic (e.g., bacterial) and eukaryotic cells, yeast, mold, and fungi. Primary cells or lines maintained in culture can be used. Furthermore, applicants envision that biological assays on viruses can be performed by infecting or transforming cells with virus. For example, and not by way of limitation, the ability of a ligand to inhibit lysogenic activity of lambda bacteriophage can be assayed by identifying transfected E. coli colonies that do not form clear plaques when infected.

Methods of the present invention for assaying activity of a synthetic test compound molecule of a library are not limited to the foregoing examples; any assay system can be modified to incorporate the presently disclosed invention are useful.

C. Enzyme Mimics/Enzyme Inhibitors

The present invention further comprises libraries that are capable of catalyzing reactions, i.e., enzyme libraries; libraries of molecules that serve as co-enzymes; and libraries of molecules that can inhibit enzyme reactions. Thus, the present invention also provides methods to be used to assay for enzyme or co-enzyme activity, or for inhibition of enzyme activity.

Enzyme activity can be observed by formation of a detectable reaction product. In a particular embodiment, an enzyme from an enzyme library catalyzes the reaction catalyzed by alkaline phosphatase, e.g., hydrolysis of 5-bromo-4-chloro-3-indoyl phosphate (BCIP) and forms a blue, insoluble reaction product on the solid phase support.

It is well known to one of ordinary skill in the art that a synthetic test compound molecule that demonstrates enzyme activity, co-enzyme activity, or that inhibits enzyme activity, can be a peptide, a peptide mimetic, or one of a variety of small-molecule compounds.

D. Topological Segregation

The present invention further encompasses a method of segregating the coding molecules and synthetic test compounds in the interior of the solid support and the crosslinker on the exterior. The method encompasses the steps of synthesizing a linker, which in the preferred embodiment is a peptide. The linker contains a sequence which can be cleaved by methods known to one of skill in the art.

V. Therapeutic and Diagnostic Agents using Compounds of the Present Invention

Once a molecular structure of interest has been identified through library screening and structural analysis of active ligands, the present invention provides molecules that comprise the molecular structure for use in treatment or diagnosis of disease. The molecule identified through screening alone can provide a diagnostic or therapeutic agent, or can be incorporated into a larger molecule. A molecule comprising a structure with biological or binding activity can be termed an “effector molecule.” The present invention further provides libraries for use in various applications. The “effector” function of the effector molecule can be any of the functions described herein or known in the art.

The method described herein not only provides a new tool to search for specific ligands of potential diagnostic or therapeutic value, but also provides important information on a series of ligands of potentially vastly different structure which nonetheless are able to interact with the same acceptor molecule. Integrating such information with molecular modeling and modern computational techniques is likely to provide new fundamental understanding of ligand-receptor interactions.

The therapeutic agents of the present invention comprise effector molecules that will bind to the biologically active site of cytokines, growth factors, or hormonal agents and thereby enhance or neutralize their action, and that will block or enhance transcription and/or translation.

The therapeutic agents of the present invention include, for example, effector molecules that bind to a receptor of pharmacologic interest such as growth factor receptors, neurotransmitter receptors, or hormone receptors. These effector molecules can be used as either agonists or antagonists of the action of the natural receptor ligand.

Another application of effector molecules that bind to receptors would be to use the binding to block the attachment of viruses or microbes that gain access to a cell by attaching to a normal cellular receptor and being internalized. Examples of this phenomenon include the binding of the human immunodeficiency virus to the CD4 receptor, and of the herpes simplex virus to the fibroblast growth factor receptor. Effector molecules that occupy the receptor could be used as pharmacologic agents to building block viral infection of target cells. Parasite invasion of cells could be similarly inhibited, after suitable effector molecules were identified according to this invention.

In another embodiment, an effector molecule comprising a structure that binds to an acceptor molecule of interest can be used to target a drug or toxin. In a preferred embodiment, the acceptor molecule of interest is a receptor or antigen found on the surface of a tumor cell, animal parasite, or microbe, e.g., bacterium, virus, unicellular parasite, unicellular pathogen, fungus or mold. In another embodiment, the targeted entity is an intracellular receptor. In yet another embodiment, an effector molecule can be an enzyme inhibitor, e.g. an inhibitor for HIV protease will be an anti-HIV agent, and a Factor Xa inhibitor will be an anti-coagulant.

In addition, it is possible that a few of the millions of synthetic test compound molecules in the pool can provide structures that have biological activity. One can isolate molecules that possess antitumor, anti-animal parasite, or antimicrobial, e.g., anti-weed, anti-plant parasite, antifungal, antibacterial, anti-unicellular parasite, anti-unicellular pathogen, or antiviral activities. In addition, some of these ligands can act as agonists or antagonists of growth factors, e.g., erythropoietin, epidermal growth factor, fibroblast growth factor, tumor growth factors, to name but a few, as well as hormones, neurotransmitters, agonists for the receptors, immunomodulators, or other regulatory molecules.

The therapeutic agents of the present invention also include effector molecules comprising a structure that has a high affinity for drugs, e.g., digoxin, benzodiazepam, heroine, cocaine, or theophylline. Such molecules can be used as an antidote for overdoses of such drugs. Similarly, therapeutic agents include effector molecules that bind to small molecules or metal ions, including heavy metals. Molecules with high affinity for bilirubin will be useful in treatment of neonates with hyperbilirubinemea.

In general, methods to identify molecules for therapy of diseases or illnesses such as are listed in the Product Category Index of The Physicians Desk Reference (PDR, 1993, 47th Edition, Medical Economics Data: Oradell, N.J., pp. 201-202) are useful. For example, an effector molecule with anti-cancer, antiparasite, anticoagulant, anticoagulant antagonist, antidiabetic agent, anticonvulsant, antidepressant, antidiarrheal, antidote, antigonadotropin, antihistamine, antihypertensive, antiinflammatory, antinauseant, antimigraine, antiparkinsonism, antiplatelet, antipruritic, antipsychotic, antipyretic, antitoxin (e.g., antivenin), bronchial dilator, vasodilator, chelating agent, contraceptive, muscle relaxant, antiglaucomatous agent, or sedative activity can be identified.

The therapeutic agents of the present invention can also contain appropriate pharmaceutically acceptable carriers, diluents and adjuvants. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like. Suitable pharmaceutical carriers are described in “Remington's Pharmaceutical Sciences” by E. W. Martin. Such compositions will contain an effective therapeutic amount of the active compound together with a suitable amount of carrier so as to provide the form for proper administration to the patient. While intravenous injection is a very effective form of administration, other modes can be employed, such as by injection, or by oral, nasal or parenteral administration.

A molecule comprising a structure determined according to the present invention can also be used to form diagnostic agents. The diagnostic agent can also be a molecule comprising one or more structures identified as a result of library screening, e.g., more than one polyamide sequence or polyalkane sequence. In addition, the diagnostic agent can contain any of the carriers described above for therapeutic agents.

As used herein, “diagnostic agent” refers to an agent that can be used for the detection of conditions such as, but not limited to, cancer such as T or B cell lymphoma, and infectious diseases as set forth above. Detection is used in its broadest sense to encompass indication of existence of condition, location of body part involved in condition, or indication of severity of condition. For example, a peptide-horseradish immunoperoxidase complex or related immunohistochemical agent could be used to detect and quantitate specific receptor or antibody molecules in tissues, serum or body fluids. Diagnostic agents can be suitable for use in vitro or in vivo. Particularly, the present invention will provide useful diagnostic reagents for use in immunoassays, Southern or Northern hybridization, and in situ assays.

In addition, the diagnostic agent can contain one or more markers such as, but not limited to, radioisotope, fluorescent tags, paramagnetic substances, or other image enhancing agents. Those of ordinary skill in the art would be familiar with the range of markers and methods to incorporate them into the agent to form diagnostic agents.

The therapeutic agents and diagnostic agents of the instant invention can be used for the treatment and/or diagnosis of animals, and more preferably, mammals including humans, dogs, cats, horses, cows, pigs, guinea pigs, mice and rats. Therapeutic or diagnostic agents can also be used to treat and/or diagnose plant diseases.

The diseases and conditions amenable to therapy or diagnosis with molecules discovered according to the present invention are as varied and wide-ranging as the permutations of structures in a library.

In another embodiment, low affinity-binding beads can be selected, and a limited library prepared based on the structure of the ligands on the beads. In another embodiment, a custom low affinity or high affinity support comprising one or a few ligands identified from the millions of synthetic test compound provided by the present invention can be used in chromatographic separations.

### VI. EXAMPLES

### Example 1

### Preparation of Bead Aggregates

Two kinds of the spatially segregated bifunctional beads (Scheme 3), one with 90% Fmoc-inside/10% NH₂-outside (compound beads), and the other with 90% Alloc-inside/10% NH₂-outside (coding beads) were prepared according to the procedure published in our laboratory (Liu, R., et al. J Am Chem Soc 2002, 124, 7678-7680). The coding beads swollen previously in DMF were treated with activated charcoal in water to yield black colored beads. The two population of beads (tan and dark) were then mixed in a ratio of 95/5, washed with water and treated with a 50% aqueous solution of glutaraldehyde, and compressed for 30 minutes inside a 20 mL syringe fitted with a frit and a detachable head on one end. The head of the syringe was detached and the formed bead aggregate block was pushed out. The bead aggregate block was then sliced into smaller pieces with a sharp razor blade to a desirable size. Each bead aggregate can carry approximately 1 μmol of compound according to the quantitative Fmoc substitution assay.

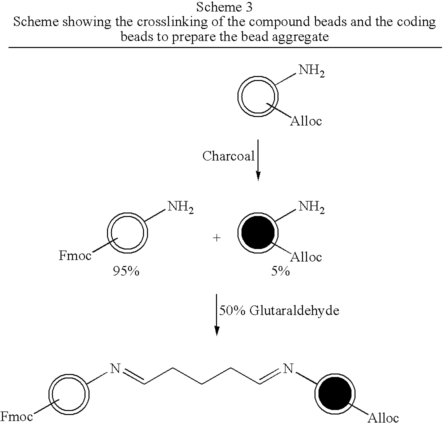

### Example 2

### Synthesis of Model Encoded Compound on Bead Aggregates

3-Isobutyl-4-benzyl-7-carbamoyl-1,2,3,4-tetrahydroquinoxalin-2-one with peptide encoding Tyr-Ile-TentaGel beads was synthesized on a sample of the bead aggregates (5 bead aggregates, which is equivalent to approximately 7 μmol of compound). The following reactions were carried out in a 5 mL polypropylene tube equipped with screw cap and the solvents were simply decanted during washing. The synthetic procedure was adopted (Lee, J., et al. J Org Chem 1997, 62, 3874-3879) without major changes and standard Fmoc based methodology was used for constructing the encoding peptide chain (Scheme 4).

Attachment of the scaffold linker (Rink-MBHA). The Fmoc protecting group from the compound beads (colorless beads) in a bead aggregate (Scheme 3) was removed by treatment with 20% piperidine in DMF at RT for 20 min. The bead aggregates were washed with DMF (3×2 mL), MeOH (3×2 mL) and DMF (3×2 mL). The solution of p-[(R,S)-α-1-(9H-Fluoren-9-yl)-methoxyformamido]-2,4-dimethoxy-benzyl-phenoxy-acetic acid (Rink-MBHA linker) 23 mg (0.042 mmol), PyBOP 22 mg (0.042) and DIEA 15 μL (0.084) in DMF were added to the bead aggregates and the mixture was shaken gently for 24 h. The bead aggregates were then washed with DMF (3×1 mL).

Attachment of the scaffold (4-Fluoro-3-nitrobenzoic acid) to bead aggregate. The Fmoc protecting group was removed by 20% piperidine in DMF at RT and the bead aggregates were washed with DMF (3×2 mL), MeOH (3×2 mL) and DMF (3×2 mL). A solution of 4-Fluoro-3-nitrobenzoic acid 8 mg (0.042 mmol), HATU 16 mg (0.042 mmol), DIEA 15 μl (0.084 mmol) in DMF 2 ml was gently mixed with the bead aggregates at RT for 24 h. The bead aggregates were then washed with DMF (3×2 mL).

Addition of first scaffold building block. A solution of H-Leu-OEt hydrochloride 22 mg (0.14 mmol), DIEA 50 μl (0.28 mmol) in DMF 3 ml was added to the bead aggregates and the mixture was shaken gently for 3 days, and the bead aggregates were washed with DMF (3×2 mL).

Encoding of first scaffold building block. The allyloxycarbonyl protecting group from coding beads (black colored) was removed by treatment with Pd[PPh₃]₄ 4 mg (0.003 mmol), PhSiH₃ 10 μL (0.08 mmol) in DCM 2 mL under an argon atmosphere at RT for 30 min. The bead aggregates were washed thoroughly with DMF (6×2 mL), water (3×2 mL), and DMF (3×1 mL). Then the solution of the coding building block (Fmoc-Leu-OH) 15 mg (0.042 mmol), DIC 7 μL (0.042 mmol), HOBt 7 mg (0.042 mmol) in DMF 2 mL was added, and the mixture was shaken gently at RT for 3 h. The bead aggregates were washed with DMF (3×2 mL).

Reduction of aryl nitro group and cyclization. The bead aggregates were treated with the solution of SnCl₂.2H₂O 50 mg (0.28 mmol) in DMF 2 mL at RT for 24 h. Then the bead aggregates were washed with DMF (3×2 mL)

Addition of second scaffold building block. A solution of benzylbromide 34 μL (0.28 mmol), K₂CO₃ 40 mg (0.28 mmol), DIEA 50 μL (0.28 mmol) in acetone 2 mL was added and the mixture was shaken gently at 70 ° C. for 48 h. The bead aggregates were washed with DMF (3×2 mL), water (3×2 mL) and DMF (3×2 mL).

Encoding of second scaffold building block. The Fmoc protecting group was removed by 20% piperidine in DMF at RT and the bead aggregates were washed with DMF (3×2 mL), MeOH (3×2 mL) and DMF (3×2 mL). To the beads bead aggregates was added the solution of the second coding building block (Boc-Tyr(tBu)-OH) 12 mg (0.042 mmol), DIC 7 μL (0.042 mmol), HOBt 7 mg (0.042 mmol) in DMF 2 mL and the mixture was shaken gently at RT for 3 h. The bead aggregates were washed with DMF (3×2 mL).

Cleavage of the compound and coding sequence deprotection. The bead aggregates were washed with DCM (3×2 mL) and treated with 10% TFA in DCM at RT for 1 h. The DCM/TFA was evaporated and the structure of the product was confirmed on HPLC (80% purity) and MS MALDI (,n/z): 338.2 (M⁺, calcd. for C₂₀H₂₃O₂N₃: 337.5). One bead aggregate was crumbled and one black bead submitted for Edman sequencing analysis. The expected sequence Tyr-Ile was found. The loading capacity of each bead aggregate tested was about 1 μmol. The size of the bead aggregate can be increased ˜7.5 fold without compromising the synthetic efficiency.

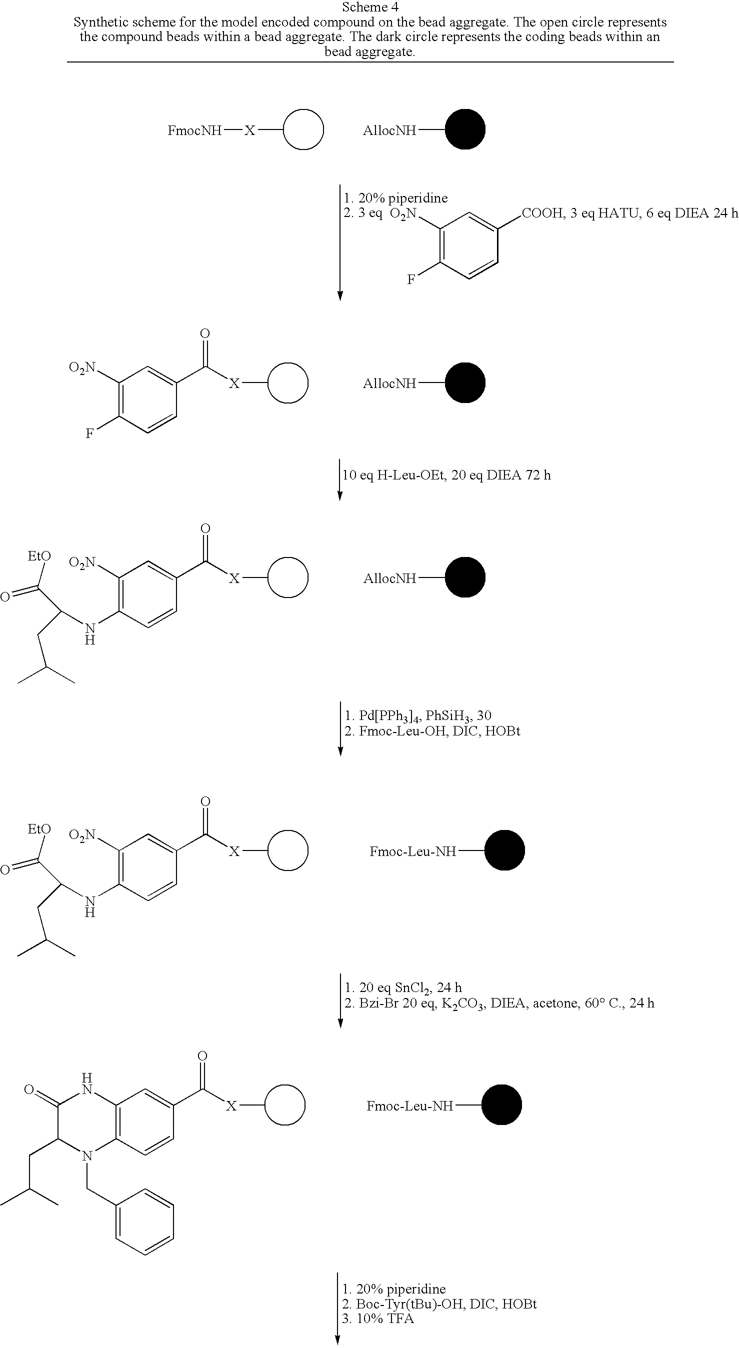

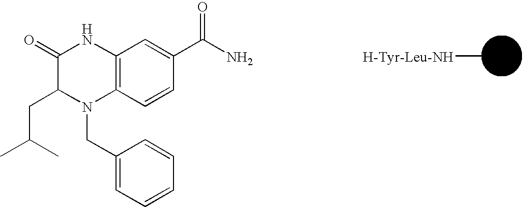

Although the foregoing invention has been described in some detail by way of illustration and example for purposes of clarity of understanding, it will be obvious that certain changes and modifications can be practiced within the scope of the appended claims. In addition, each reference provided herein is incorporated by reference in its entirety to the same extent as if each reference was individually incorporated by reference.

What is claimed is:

1. A method for preparing a library of compounds, comprising:
a) providing a plurality of individual bead aggregates, wherein each of said bead aggregates comprises a population of compound beads and a population of coding beads, wherein said compound beads and said coding beads are crosslinked to each other, wherein each of said compound beads comprises a scaffold linked to said compound bead via a scaffold linker, and with at least two scaffold functional groups attached to said scaffold, and wherein each of said coding beads comprises at least one coding functional group;
b) contacting a first bead aggregate with a first reactive component such that a first scaffold functional group reacts with said first reactive component to afford a first scaffold building block;
c) contacting said first bead aggregate with a successive reactive component such that a subsequent scaffold functional group reacts with said successive reactive component to afford a subsequent scaffold building block;
d) repeating step c) until said first compound has been prepared; and
e) subjecting additional bead aggregates to steps b)-d) with additional reactive components to prepare said library of compounds.

2. The method of claim 1, further comprising the following step:
f) cleaving each of said compounds from each of said bead aggregates.

3. The method of claim 1, wherein said reactive component is attached via a reaction selected from the group consisting of amine acylation, reductive alkylation, aromatic reduction, aromatic acylation, aromatic cyclization, aryl-aryl coupling, [3+2] cycloaddition, Mitsunobu reaction, nucleophilic aromatic substitution, sulfonylation, aromatic halide displacement, Michael addition, Wittig reaction, Knoevenagel condensation, reductive amination, Heck reaction, Stille reaction, Suzuki reaction, Aldol condensation, Claisen condensation, amino acid coupling, amide bond formation, acetal formation, Diels-Alder reaction, [2+2] cycloaddition, enamine formation, esterification, Friedel Crafts reaction, glycosylation, Grignard reaction, Horner-Emmons reaction, hydrolysis, imine formation, metathesis reaction, nucleophilic substitution, oxidation, Pictet-Spengler reaction, Sonogashira reaction, thiazolidine formation, thiourea formation and urea formation.

4. The method of claim 1, wherein the compounds of said library are prepared in parallel.

5. The method of claim 1, wherein said bead aggregates comprise units of formula I: 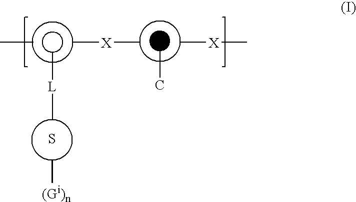
wherein
(Gⁱ)ₙ represents n independent scaffold functional groups, G¹ to Gⁿ, wherein each Gⁱ is a scaffold functional group; 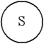
is a scaffold;
L is a scaffold linker; 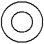
is said compound bead, wherein the inner circle represents an interior portion of said compound bead, and the outer circle represents an exterior portion of said compound bead; 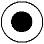
is said coding bead, wherein the darkened portion represents an interior portion of said coding bead, and the lightened portion represents an exterior portion of said coding bead;
C represents said coding functional group;
X is a crosslinker linking said compound bead to said coding bead;
subscript n is an integer from 2 to 10; and
superscript i is an integer from 1 to n.

6. The method of claim 5, wherein said bead aggregates comprise units of formula Ia:

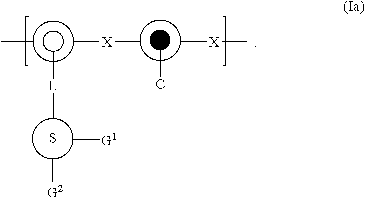

7. The method of claim 5, wherein said bead aggregates comprise units of formula Ib:

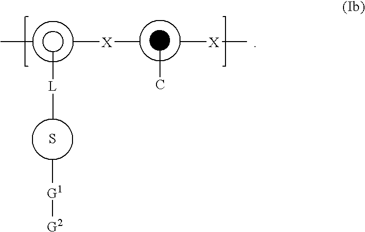

8. The method of claim 5, wherein said bead aggregates comprise units of formula Ic:

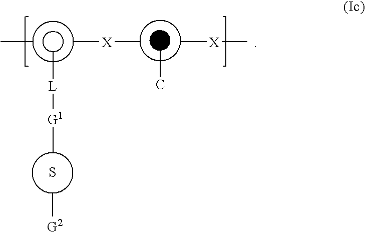

9. The method of claim 1, further comprising the step of encoding each of said scaffold building blocks with a coding building block.

10. The method of claim 9, wherein each of said scaffold building blocks is encoded with one of said coding building blocks prior to, simultaneously with, or following each of said contacting steps of claim 1.

11. The method of claim 9, wherein said steps b)-d) afford bead aggregates comprised of units of formula II: 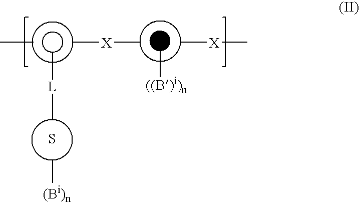
wherein
(Bⁱ)ₙ represents n independent scaffold building blocks, B¹ to Bⁿ, wherein each Bⁱ is a scaffold building block; 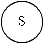
is a scaffold;
L is a scaffold linker; 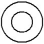
is said compound bead, wherein the inner circle represents an interior portion of said compound bead, and the outer circle represents an exterior portion of said compound bead; 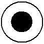
is said coding bead, wherein the darkened portion represents an interior portion of said coding bead, and the lightened portion represents an exterior portion of said coding bead;
X is a crosslinker linking said compound bead to said coding bead;
subscript n is an integer from 2 to 10; and
superscript i is an integer from 1 to n.

12. The method of claim 11, wherein said encoding step occurs following said contacting step.

13. The method of claim 12, wherein subsequent coding building blocks are attached to said coding bead via previously attached coding building blocks.

14. The method of claim 13, wherein said bead aggregates comprise units of formula Ia: 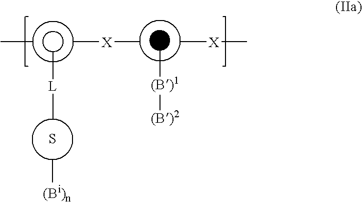
wherein subscript n is 2.

15. The method of claim 11, wherein said encoding step is performed simultaneously with said contacting step.

16. The method of claim 15, wherein each of said coding building blocks is separately attached to said coding bead.

17. The method of claim 16, wherein said bead aggregates comprise units of formula IIb: 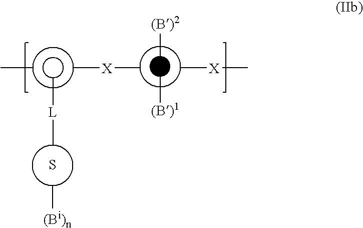
wherein subscript n is 2.

18. The method of claim 1, wherein said compound beads and said coding beads are present in each of said bead aggregates in a ratio of 99.9/0.1 to 50.0/50.0.

19. The method of claim 1, wherein said scaffold is the same on each of said bead aggregates.

20. The method of claim 1, wherein at least two different scaffolds are used.

21. The method of claim 1, wherein said library of compounds is prepared via a split-mix methodology.

22. A library of compounds prepared by the method of claim 1.

23. A library of compounds prepared by the method of claim 2.

24. A method for identifying a compound of claim 2 that binds to a target, said method comprising:
a) contacting said compound of claim 2 with said target; and
b) determining the functional effect of said compound upon said target.

25. A method for preparing a library of compounds, comprising:
a) providing a plurality of individual bead aggregates, wherein each of said bead aggregates comprises a population of compound beads and a population of coding beads, wherein said compound beads and said coding beads are crosslinked to each other, wherein each of said compound beads comprises a scaffold linked to said compound bead via a scaffold linker, and with at least two scaffold functional groups attached to said scaffold, and wherein each of said coding beads comprises at least one coding functional group;
b) splitting said bead aggregates into two or more separate pools;
c) contacting said bead aggregates with one or more first reactive components in said two or more separate pools such that a first scaffold functional group reacts with one of said first reactive components to afford a first scaffold building block, wherein said contacting step affords subsequent bead aggregates;
d) encoding each of said scaffold building blocks with a coding building block, comprising the step of contacting said coding functional group with a reactive component such that said coding functional group reacts with said reactive component to afford a coding building block linked to said coding bead, wherein said coding building block encodes one of said scaffold building blocks, and wherein said encoding step yields subsequent encoded bead aggregates;
e) mixing said subsequent encoded bead aggregates from said two or more separate pools into a single pool;
f) splitting said subsequent encoded bead aggregates into two or more separate pools;
g) contacting said subsequent encoded bead aggregates in said two or more separate pools with a successive reactive component such that a subsequent scaffold functional group reacts with said successive reactive component to afford a subsequent scaffold building block, wherein said contacting step yields further bead aggregates;
h) repeating step d), wherein said encoding step yields further encoded bead aggregates;
i) repeating steps e)-h), wherein said further encoded bead aggregates of step h) become said subsequent encoded bead aggregates of step e), until said library of compounds has been prepared.
